SEQUENCE LISTING (57 sequences)

SEQ ID NO: 1 - protein, 14 aa, Artificial Sequence
  SARS-Cov2 spike protein Endogenous signal peptide
MFVFLVLLPLVSSQ

SEQ ID NO: 2 - protein, 23 aa, Artificial Sequence
  Tissue plasminogen activator signal peptide
MDAMKRGLCCVLLLCGAVFVSPS

SEQ ID NO: 3 - protein, 4 aa, Artificial Sequence
  Flexible linker
GGGS

SEQ ID NO: 4 - protein, 4 aa, Artificial Sequence
  Flexible linker
GGGP

SEQ ID NO: 5 - protein, 5 aa, Artificial Sequence
  Flexible linker
GGSGG

SEQ ID NO: 6 - protein, 8 aa, Artificial Sequence
  Flexible linker
GGSGGGGS

SEQ ID NO: 7 - protein, 8 aa, Artificial Sequence
  His tag (8x)
HHHHHHHH

SEQ ID NO: 8 - protein, 10 aa, Artificial Sequence
  His tag (10x)
HHHHHHHHHH

SEQ ID NO: 9 - protein, 214 aa, Artificial Sequence
  Murine IgG1-Fc (CH2, CH3 only) tag (without hinge)
VPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGI

SEQ ID NO: 10 - protein, 227 aa, Artificial Sequence
  Murine IgG1-Fc Dimerization domain
VPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGI

SEQ ID NO: 11 - protein, 210 aa, Artificial Sequence
  Murine IgG-2a-Fc (CH2, CH3 only) tag (without hinge)
PSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 12 - protein, 232 aa, Artificial Sequence
  Murine IgG2a-Fc Dimerization domain
PRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 13 - protein, 40 aa, Artificial Sequence
  p53 Tetramerization domain
KPLDGEYFTLQIRGRERFEMFRELNEALELKDAQAGKEPG

SEQ ID NO: 14 - protein, 124 aa, Artificial Sequence
  SARS-CoV2 nucleocapsid N-terminal domain
ASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYA

SEQ ID NO: 15 - protein, 101 aa, Artificial Sequence
  SARS-CoV2 nucleocapsid C-terminal domain
AEASKKNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTF

SEQ ID NO: 16 - protein, 13 aa, Artificial Sequence
  Murine IgG1-Fc Hinge Domain
VPRDCGCKPCICT

SEQ ID NO: 17 - protein, 38 aa, Artificial Sequence
  Streptavidin binding peptide tag
MDEKTTGWRGGHVVEGLAGELEQLRARLEHHPQGQREP

SEQ ID NO: 18 - protein, 308 aa, Artificial Sequence
  HRP enzyme
QLTPTFYDNSCPNVSNIVRDTIVNELRSDPRIAASILRLHFHDCFVNGCDASILLDNTTSFRTEKDAFGNANSARGFPVIDRMKAAVESACPRTVSCADLLTIAAQQSVTLAGGPSWRVPLGRRDSLQAFLDLANANLPAPFFTLPQLKDSFRNVGLNRSSDLVALSGGHTFGKNQCRFIMDRLYNFSNTGLPDPTLNTTYLQTLRGLCPLNGNLSALVDFDLRTPTIFDNKYYVNLEEQKGLIQSDQELFSSPNATDTIPLVRSFANSTQTFFNAFVEAMDRMGNITPLTGTQGQIRLNCRVVNSNS

SEQ ID NO: 19 - protein, 6 aa, Artificial Sequence
  TEV Cleavage site
ENLYFQ

SEQ ID NO: 20 - protein, 223 aa, Artificial Sequence
  RBD
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 21 - protein, 255 aa, Artificial Sequence
  pxENB14-RBD
MFVFLVLLPLVSSQHHHHHHHHGGGSENLYFQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 22 - protein, 249 aa, Artificial Sequence
  pxENB17-RBD
MFVFLVLLPLVSSQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGGGSHHHHHHHH

SEQ ID NO: 23 - protein, 264 aa, Artificial Sequence
  pxENB15-RBD
MDAMKRGLCCVLLLCGAVFVSPSHHHHHHHHGGGSENLYFQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 24 - protein, 258 aa, Artificial Sequence
  pxENB18-RBD
MDAMKRGLCCVLLLCGAVFVSPSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGGGSHHHHHHHH

SEQ ID NO: 25 - protein, 473 aa, Artificial Sequence
  pxEBNCP21-RBD
MDAMKRGLCCVLLLCGAVFVSPSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGI

SEQ ID NO: 26 - protein, 478 aa, Artificial Sequence
  pxEBNCP22-RBD
MDAMKRGLCCVLLLCGAVFVSPSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 27 - protein, 464 aa, Artificial Sequence
  pxEBNCP23-RBD
MDAMKRGLCCVLLLCGAVFVSPSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGGGPVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGI

SEQ ID NO: 28 - protein, 460 aa, Artificial Sequence
  pxEBNCP24-RBD
MDAMKRGLCCVLLLCGAVFVSPSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGGGPPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 29 - protein, 298 aa, Artificial Sequence
  pxEBNCP25-RBD
MDAMKRGLCCVLLLCGAVFVSPSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGGGPKPLDGEYFTLQIRGRERFEMFRELNEALELKDAQAGKEPGHHHHHHHH

SEQ ID NO: 30 - protein, 285 aa, Artificial Sequence
  pxEBNCP29-RBD
MDAMKRGLCCVLLLCGAVFVSPSVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKGGGSHHHHHHHH

SEQ ID NO: 31 - protein, 222 aa, Artificial Sequence
  pxEBNCP30-RBD
MDAMKRGLCCVLLLCGAVFVSPSITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLGGGSHHHHHHHH

SEQ ID NO: 32 - protein, 261 aa, Artificial Sequence
  pxEBNCP31-RBD
MDAMKRGLCCVLLLCGAVFVSPSITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKGGGSHHHHHHHH

SEQ ID NO: 33 - protein, 389 aa, Artificial Sequence
  pxENBEP32-NucRBD
MDAMKRGLCCVLLLCGAVFVSPSASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAGGSGGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGGGSHHHHHHHHHH

SEQ ID NO: 34 - protein, 389 aa, Artificial Sequence
  pxENBEP33-RBDNuc
MDAMKRGLCCVLLLCGAVFVSPSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGGSGGASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAGGGSHHHHHHHHHH

SEQ ID NO: 35 - protein, 498 aa, Artificial Sequence
  pxENBEP34-NucRBD
MDAMKRGLCCVLLLCGAVFVSPSASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAGGSGGGGSAEASKKNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFGGSGGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGGGSHHHHHHHHHH

SEQ ID NO: 36 - protein, 498 aa, Artificial Sequence
  pxENBEP35-RBDNuc
MDAMKRGLCCVLLLCGAVFVSPSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGGSGGASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAGGSGGGGSAEASKKNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFGGGSHHHHHHHHHH

SEQ ID NO: 37 - protein, 597 aa, Artificial Sequence
  pxEBNCP26-RBD
MDAMKRGLCCVLLLCGAVFVSPSVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKGGGSQLTPTFYDNSCPNVSNIVRDTIVNELRSDPRIAASILRLHFHDCFVNGCDASILLDNTTSFRTEKDAFGNANSARGFPVIDRMKAAVESACPRTVSCADLLTIAAQQSVTLAGGPSWRVPLGRRDSLQAFLDLANANLPAPFFTLPQLKDSFRNVGLNRSSDLVALSGGHTFGKNQCRFIMDRLYNFSNTGLPDPTLNTTYLQTLRGLCPLNGNLSALVDFDLRTPTIFDNKYYVNLEEQKGLIQSDQELFSSPNATDTIPLVRSFANSTQTFFNAFVEAMDRMGNITPLTGTQGQIRLNCRVVNSNSGGGSHHHHHHHH

SEQ ID NO: 38 - protein, 597 aa, Artificial Sequence
  pxEBNCP27-RBD
MDAMKRGLCCVLLLCGAVFVSPSHHHHHHHHGGGSQLTPTFYDNSCPNVSNIVRDTIVNELRSDPRIAASILRLHFHDCFVNGCDASILLDNTTSFRTEKDAFGNANSARGFPVIDRMKAAVESACPRTVSCADLLTIAAQQSVTLAGGPSWRVPLGRRDSLQAFLDLANANLPAPFFTLPQLKDSFRNVGLNRSSDLVALSGGHTFGKNQCRFIMDRLYNFSNTGLPDPTLNTTYLQTLRGLCPLNGNLSALVDFDLRTPTIFDNKYYVNLEEQKGLIQSDQELFSSPNATDTIPLVRSFANSTQTFFNAFVEAMDRMGNITPLTGTQGQIRLNCRVVNSNSGGGSVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNK

SEQ ID NO: 39 - protein, 300 aa, Artificial Sequence
  pxENB36-H8RBDgpp53
MFVFLVLLPLVSSQCHHHHHHHHGGGSENLYFQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGGGPKPLDGEYFTLQIRGRERFEMFRELNEALELKDAQAGKEPG

SEQ ID NO: 40 - protein, 300 aa, Artificial Sequence
  pxENB37-H8RBDgsp53
MFVFLVLLPLVSSQCHHHHHHHHGGGSENLYFQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGGGSKPLDGEYFTLQIRGRERFEMFRELNEALELKDAQAGKEPG

SEQ ID NO: 41 - protein, 290 aa, Artificial Sequence
  pxENB38-RBDgpp53H8
MFVFLVLLPLVSSQCRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGGGPKPLDGEYFTLQIRGRERFEMFRELNEALELKDAQAGKEPGHHHHHHHH

SEQ ID NO: 42 - protein, 290 aa, Artificial Sequence
  pxENB39-RBDgsp53H8
MFVFLVLLPLVSSQCRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGGGSKPLDGEYFTLQIRGRERFEMFRELNEALELKDAQAGKEPGHHHHHHHH

SEQ ID NO: 43 - protein, 483 aa, Artificial Sequence
  pxENB40-H8RBDFc
MFVFLVLLPLVSSQCHHHHHHHHGGGSENLYFQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 44 - protein, 525 aa, Artificial Sequence
  pxENB41-H8RBDFcSBP
MFVFLVLLPLVSSQCHHHHHHHHGGGSENLYFQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGKGGGSMDEKTTGWRGGHVVEGLAGELEQLRARLEHHPQGQREP

SEQ ID NO: 45 - protein, 269 aa, Artificial Sequence
  pxENB42-H8RBDFcHG
MFVFLVLLPLVSSQCHHHHHHHHGGGSENLYFQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFVPRDCGCKPCICT

SEQ ID NO: 46 - protein, 311 aa, Artificial Sequence
  pxENB43-RBDFcHGSBP
MFVFLVLLPLVSSQCHHHHHHHHGGGSENLYFQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFVPRDCGCKPCICTGGGSMDEKTTGWRGGHVVEGLAGELEQLRARLEHHPQGQREP

SEQ ID NO: 47 - protein, 479 aa, Artificial Sequence
  pxENB44-H8RBDRBD
MFVFLVLLPLVSSQCHHHHHHHHGGGSENLYFQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 48 - protein, 473 aa, Artificial Sequence
  pxENB46-RBDRBDH8
MFVFLVLLPLVSSQCRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGGGSHHHHHHHH

SEQ ID NO: 49 - protein, 298 aa, Artificial Sequence
  pxENB48-H8RBDSBP
MFVFLVLLPLVSSQCHHHHHHHHGGGSENLYFQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGGGSMDEKTTGWRGGHVVEGLAGELEQLRARLEHHPQGQREP

SEQ ID NO: 50 - protein, 255 aa, Artificial Sequence
  pxENB14-RBD-B.1.1.7
MFVFLVLLPLVSSQHHHHHHHHGGGSENLYFQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNF

SEQ ID NO: 51 - protein, 255 aa, Artificial Sequence
  pxENB14-RBD-B.1.351
MFVFLVLLPLVSSQHHHHHHHHGGGSENLYFQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNF

SEQ ID NO: 52 - protein, 255 aa, Artificial Sequence
  pxENB14-RBD-B.1.617.2
MFVFLVLLPLVSSQHHHHHHHHGGGSENLYFQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNF

SEQ ID NO: 53 - protein, 255 aa, Artificial Sequence
  pxENB14-RBD-B.1.427
MFVFLVLLPLVSSQHHHHHHHHGGGSENLYFQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 54 - protein, 255 aa, Artificial Sequence
  pxENB14-RBD-P.1
MFVFLVLLPLVSSQHHHHHHHHGGGSENLYFQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNF

SEQ ID NO: 55 - protein, 473 aa, Artificial Sequence
  pxENB46-RBD2-B.1.1.7
MFVFLVLLPLVSSQCRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFGGGSHHHHHHHH

SEQ ID NO: 56 - protein, 473 aa, Artificial Sequence
  pxENB46-RBD2-B.1.351
MFVFLVLLPLVSSQCRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFGGGSHHHHHHHH

SEQ ID NO: 57 - protein, 473 aa, Artificial Sequence
  pxENB46-RBD2-B.1.617.2
MFVFLVLLPLVSSQCRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFGGGSHHHHHHHH

(19) United States
(12) Patent Application Publication
(10) Pub. No.: US 2024/0270795 A1
(43) Pub. Date: Aug. 15, 2024

(54) FUSION PROTEINS COMPRISING SARS-COV-2 RECEPTOR BINDING DOMAIN

(71) Applicant: GRIFOLS DIAGNOSTIC SOLUTIONS INC., Emeryville, CA (US)

(72) Inventors: ELISABETE NASCIMENTO, Emeryville, CA (US); ELIZABETH A. BOOTH, Emeryville, CA (US); EFRAIN CEH PAVIA, Emeryville, CA (US); TRISTAN WASLEY, Emeryville, CA (US); ZEBULON LAPOINT, Emeryville, CA (US); CHARLES HOLZ, Emeryville, CA (US); ROBYN CASSAN, Emeryville, CA (US); COLLEEN BROWN, Emeryville, CA (US); Jody MELTON WITT, Emeryville, CA (US)

(21) Appl. No.: 18/020,870

(22) PCT Filed: Aug. 17, 2021
(86) PCT No.: PCT/IB2021/057546

§ 371 Date: Feb. 10, 2023

Related U.S. Application Data
(60) Provisional application No. 63/066,684, filed on Aug. 17, 2020.

(51) Int. Cl. C07K 14/005; C12N 9/72

(52) U.S. Cl. CPC C07K 14/005; C12N 9/6459; C07K 2319/02; C07K 2319/21; C07K 2319/22; C07K 2319/30; C07K 2319/50; C07K 2319/61; C07K 2319/70; C12N 2770/20022

(57) ABSTRACT

A fusion protein includes the SARS-COV-2 receptor binding domain (RBD) of the SARS-COV-2 spike protein or a fragment, and a N-terminal signal peptide, and at least one of the following: a polyhistidine tag, linker, an oligomerization tag, a region in spike protein outside RBD, a horseradish peroxidase binding domain or a protease cleavage site.

### PRIORITY AND CROSS REFERENCE TO RELATED APPLICATIONS

This application is the U.S. National Phase Application under 35 U.S.C. § 371 of International Application No. PCT/IB2021/057546, filed Aug. 17, 2021, designating the U.S. and published as WO 2022/038501 A1 on Feb. 24, 2022, which claims the benefit of Provisional Application No. 63066684, filed Aug. 17, 2020. Any and all applications for which a foreign or a domestic priority is claimed is/are identified in the Application Data Sheet filed herewith and is/are hereby incorporated by reference in their entireties under 37 C.F.R. § 1.57.

### SEQUENCE LISTING

This application is being filed along with a Sequence Listing in electronic format. The Sequence Listing is provided as a file entitled SeqList-DURC048.012APC, created Jan. 23, 2023, which is approximately 85 kilobytes in size, which is replaced by a Replacement Electronic Sequence Listing submitted herewith as a file entitled HOFM028001APCREPLACEMENTSEQLIST.txt, which is 138,440 bytes in size and was created on Aug. 14, 2023. The information in the electronic format of the Sequence Listing is incorporated herein by reference in its entirety.

### FIELD

This application relates to the medical field of COVID-19 diagnosis or treatment, and in particular, it relates to fusion proteins comprising severe acute respiratory syndrome coronavirus 2 (SARS-CoV-2) receptor binding domain (RBD) or a fragment thereof. Said fusion proteins are useful for the development of assays capable of screening reagents that inhibit binding of the viral spike (S) protein to the angiotensin converting enzyme 2 (ACE2).

### BACKGROUND

SARS-COV-2 is an enveloped RNA virus from the Coronaviridae family (Gorbalenya, A.E, et al., 2020, Nature Microbiology, 5(4):p.536-544) that has several structural components, including Spike (S), Envelope (E), Membrane (M) and Nucleocapsid (N) proteins (Lu, R., et al., 2020, Lancet 395(10224): p.565-574). The S protein consists of two subunits (S1 and S2) that form a trimer on the viral membrane; S1 contains the RBD which is responsible for binding to the ACE2 host cell receptor (Hoffmann, M., et. al., 2020, Cell, 181(2):p.271-280.e8), while S2 enables the fusion between the host and viral membranes (Lan, J., et al., 2020, Nature, 581(7807):215-220; Wrapp, D., et al., 2020, Science, 367(6483) :p. 1260-1263).

SARS-COV-2 has caused a widespread COVID-19 pandemic that infected millions worldwide and claimed hundreds of thousands of lives. Currently, the main and most accurate method of diagnosis is by PCR testing of nasopharyngeal swabs (Peng et al., 2020, J Med Virol. 24;10.1002/jmv.25936); yet, there is an urgent need to develop reliable, highly sensitive and specific antibody tests capable of identifying all infected individuals, irrespective of clinical symptoms. This information will be critical to establish community surveillance and implement policies that contain the viral spread.

The US Food and Drug Administration (FDA) has granted Emergency Use Authorizations (EUA) to multiple immunoassay tests in the market, but none of those assays has been fully validated. Because of the lack of validated immunoassays, key to understand risk, epidemiological factors, pathogenesis and mortality, the present inventors developed fusion proteins that comprise RBD molecular designs aimed at being a reagent in SARS-COV-2 immunoassays.

The spike RBD represents a promising antigen for the detection of anti-SARS-COV-2 IgGs aimed at identifying current and past infections; and because the RBD is poorly conserved among other SARS-CoVs and pathogenic human coronavirus, it shows an enhanced capacity to recognize total anti-SARS-COV-2 Igs and IgMs (Premkumar, L. et al., 2020, Science Immunology, (10):p1126-1140). The concerns of lower assay sensitivity due to the small size of the RBD protein may be overcome by the molecular fusion of RBD and N proteins. The goal of this invention is to improve assay specificity (RBD truncations and RBD mutations) and sensitivity (RBD-N fusions, RBD-multimerization domains; RBD-horseradish peroxidase (HRP)).

The inventors of the present invention have developed RBD fusion proteins and molecular designs that facilitate the identification of hyperimmune human sera to be used as a therapeutic or for therapeutic development. A large fraction of antibodies developed against RBD show neutralizing properties, the rationale being that these mAbs disrupt the interaction between S and hACE2 proteins, preventing viral entry. As of Jun. 29, 2020 the FDA has not approved convalescent plasma therapy, however it recommends under investigational studies and clinical trials, to use a titer of at least 1:160 for human passive immunization studies. Because RBD elicits the development of antibodies with antiviral activity, these proteins will be essential for the development of inhibitory assays that identify neutralizing antibodies against SARS-COV-2.

The present invention describes a new composition of matter for the production of RBD fusion proteins. This invention embodies the methods for producing RBD fusion proteins as well as the nucleic acid molecules encoding RBD, their expression vectors and host cells. It also covers RBD truncations, multimerization domains and fusions to N protein. This novel composition of matter also embodies mutations identified by molecular dynamics simulations and affinity maturation that have been described as enhancers of expression or affinity to ACE2. The described molecular designs can be used as key reagents in antibody titer, inhibitor/neutralization screening assays, vaccine development or as agents to elicit the production of therapeutic antibodies with antiviral activity. These fusion proteins can also be fused to HRP for enabling SARS-COV-2 detection and quantification.

The present inventors have developed non-obvious RBD molecular designs containing IgG1, IgG2aFc and p53 dimerization and tetramerization domains, with the goal of increasing assay avidity and sensitivity; while also producing high quality, well characterized and reproducible material. In addition, embodiments where the RBD with N proteins are fused together were designed, as well as RBD and HRP with the goal of improving assay sensitivity during the acute phase of infection, as N protein is detected early during the infection.

The described molecules are specifically recognized by anti-SARS-COV-2 S/S1/RBD polyclonal rabbits antibodies and can be used as single entities in capturing anti-SARS-COV-2 total IgG or IgM antibodies in immunoassay platforms. When a full assay is developed, these molecules can be immobilized in a solid support such as a microtiter plate, a membrane, a bead, a polypeptide chip, or a chromatography column. A subset of the presented designs has been experimentally tested with similar or better performance (measured as affinity to hACE2) than other commercial counterparts.

Finally, due to the strong antiviral activity of RBD-specific antibodies, the RBD proteins herein described can be used as vaccine candidates to elicit broadly effective anti-SARS-COV-2 antibodies (Robbiani, D. et al., 2020, Nature, doi: https://doi.org/10.1101/2020.05.13.092619; Huo, J. et al., 2020, Cell Host & Microbe, (28):p1-10).

### SUMMARY

In a first aspect, the present invention relates to a fusion protein comprising the SARS-COV-2 receptor binding domain (RBD) of the SARS-COV-2 spike protein or a fragment thereof, and a N-terminal signal peptide, and at least one of a polyhistidine tag, a linker, an oligomerization tag, a region in spike protein outside RBD, a horseradish peroxidase binding domain or a protease cleavage site.

In one embodiment, said N-terminal signal peptide is selected from a SARS-COV-2 spike endogenous signal peptide, or a tissue plasminogen activator (tPa) signal peptide. In one, embodiment, said N-terminal signal peptide has an amino acid sequence selected from SEQ ID NO:1 and SEQ ID NO:2.

In one embodiment, said polyhistidine tag consists of 8 or 10 histidine residues. In one, embodiment, said polyhistidine tag has an amino acid sequence selected from SEQ ID NO:7 and SEQ ID NO:8.

In one embodiment, said oligomerization tag is selected from a murine IgG1-Fc (CH2, CH3 only), a murine IgG1-Fc dimerization domain, a murine IgG-2a-Fc (CH2, CH3 only), a murine IgG-2a-Fc dimerization domain, a p53 tetramerization domain, a SARS-COV-2 nucleocapsid N-terminal domain and a SARS-COV-2 nucleocapsid C-terminal domain. In one embodiment, said oligomerization tag has an amino acid sequence selected from SEQ ID NO:9, SEQ ID NO: 10, SEQ ID NO: 11, SEQ ID NO: 12, SEQ ID NO: 13, SEQ ID NO: 14 and SEQ ID NO: 15.

In one embodiment, said linker is a flexible linker. In one embodiment, said linker has an amino acid sequence selected from SEQ ID NO:3, SEQ ID NO:4, SEQ ID NO:5 and SEQ ID NO:6.

In one embodiment, the streptavidin binding peptide tag has or comprises the amino acid sequence of the SEQ ID NO: 17. In one embodiment, said horseradish peroxidase binding domain has an amino acid sequence selected from SEQ ID NO:18.

In one embodiment, said protease cleavage site is selected from a tobacco etch virus cleavage site (TEV). In one embodiment, said protease cleavage site has an amino acid sequence selected from SEQ ID NO: 19.

In one embodiment, said receptor binding domain (RBD) of the SARS-COV-2 spike protein or a fragment thereof has an amino acid sequence of at least about 90%, or at least 95% sequence identity with SEQ ID NO:20.

In one embodiment, said fusion protein has an amino acid sequence of at least 90%, or at least 95% sequence identity with SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, or SEQ ID NO:57.

In one embodiment, said SARS-COV-2 RBD protein comprises a mutation in one or more of the following positions: G404, A475, T478, N481, G485, F490, Q493, G496, Q498, N501, or V503.

In a further aspect, the present invention refers to a cell, comprising the fusion protein as described above.

In a further aspect, the present invention refers to a nucleic acid comprising a nucleotide sequence encoding the fusion protein, a promoter operably linked to the nucleotide sequence and a selectable marker.

In another aspect, the present invention refers to a cell comprising the above-mentioned nucleic acid.

Finally, the present invention refers to a composition comprising the above-mentioned fusion protein and a solid support, wherein the fusion protein is covalently or non-covalently bound to the solid support.

### BRIEF DESCRIPTION OF THE DRAWINGS

FIG. 1 shows the expression and purification of SARS-COV-2 fusion proteins. A) Schematic showing the characteristics of pxENB14-RBD (top) and pxENB17-RBD constructs (bottom). B) Average yields of pxENB14-RBD and pxENB17-RBD produced in Expi293 cells harvested at day 3, and C) Western-Blot analysis of Expi293 supernatants harvested at day 3 using anti-His tag mouse monoclonal antibody. Samples were treated under reducing conditions. D) RBD proteins were purified using Nickel affinity chromatography. E) SDS-PAGE showing apparent molecular mass and purity for pxENB14-RBD and pxENB17-RBD purifications. F) & G) SDS-PAGE of final purified samples, reduced (R) and non-reduced (NR), run on an 8-16% TGX stain free gel. M: Protein Ladder (Precision Plus Unstained Protein Standard). H) & I) Western-blot analysis using S1 Rabbit polyclonal antibody (Sino Biological) at a 1:1000 dilution.

FIG. 2 is a Cryo-EM structure of ACE2 docked to RBD. Structure was retrieved from PDB structure 6M1710. ACE2 (green). RBD (Cyan).

FIG. 3 are A) Binding of immobilized hACE2 with SARS-COV-2 RBD; Biolayer interferometry sensorgrams illustrating human ACE2 receptor-RBD interactions: B) pxENB14-His-TEV-RBD C) pxENB17-RBD and D) RBD produced from a commercial source. Data is shown in different color lines depending on analyte concentration and the data was best fitted to a 1:1 binding model as shown by the red line.

FIG. 4 are SDS-PAGEs of supernatants from Expi293 cells expressing each of the constructs depicted. All samples were reduced in the presence of DTT. Samples ran on a 8-16% TGX stain free gel. M: Protein Ladder (Precision Plus Unstained Protein Standard). Western-blot analysis using 1:1000 of anti-His mAb; SP: supernatant; PL: pellet. Arrowhead highlights protein band.

FIG. 5 are biolayer interferometry sensorgrams illustrating human ACE2 receptor-multimeric RBD protein interactions.

FIG. 6 are biolayer interferometry sensorgrams illustrating human ACE2 receptor-pxENB14 mutants.

FIG. 7 are biolayer interferometry sensorgrams illustrating human ACE2 receptor-pxENB46 mutants.

### DETAILED DESCRIPTION

Unless defined otherwise, all technical and scientific terms used herein have the same meaning as commonly understood by one of ordinary skill in the art pertinent to the methods and compositions described. As used herein, the following terms and phrases have the meanings ascribed to them unless specified otherwise.

The terms “a,” “an,” and “the” include plural referents, unless the context clearly indicates otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Unless otherwise defined, all technical and scientific terms used herein have the same meaning as commonly understood by one of ordinary skill in the art to which this invention pertains. Exemplary methods and materials are described below, although methods and materials similar or equivalent to those described herein can also be used and will be apparent to those of skill in the art. All publications and other references mentioned herein are incorporated by reference in their entirety. In case of conflict, the present specification, including definitions, will control. The materials, methods, and examples are illustrative only and not intended to be limiting.

Each embodiment in this specification is to be applied mutatis mutandis to every other embodiment unless expressly stated otherwise.

The following terms, unless otherwise indicated, shall be understood to have the following meanings:

As used herein, the term “nucleic acid” refers to any materials comprised of DNA or RNA. Nucleic acids can be made synthetically or by living cells.

As used herein, the term “protein” refers to large biological molecules, or macromolecules, consisting of one or more chains of amino acid residues. Many proteins are enzymes that catalyze biochemical reactions and are vital to metabolism. Proteins also have structural or mechanical functions, such as actin and myosin in muscle and the proteins in the cytoskeleton, which form a system of scaffolding that maintains cell shape. Other proteins are important in cell signaling, immune responses, cell adhesion, and the cell cycle. However, proteins may be completely artificial or recombinant, i.e., not existing naturally in a biological system.

As used herein, the term “polypeptide” refers to both naturally-occurring and non-naturally-occurring proteins, and fragments, mutants, derivatives and analogs thereof. A polypeptide may be monomeric or polymeric. A polypeptide may comprise a number of different domains (peptides) each of which has one or more distinct activities.

As used herein, the term “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids.

As used herein, the term “fusion protein” refers to proteins comprising two or more amino acid sequences that do not co-exist in naturally-occurring proteins. A fusion protein may comprise two or more amino acid sequences from the same or from different organisms. The two or more amino acid sequences of a fusion protein are typically in frame without stop codons between them and are typically translated from mRNA as part of the fusion protein.

The term “fusion protein” and the term “recombinant” can be used interchangeably herein.

As used herein, the term “antigen” refers to a biomolecule that binds specifically to the respective antibody. An antibody from the diverse repertoire binds a specific antigenic structure by means of its variable region interaction.

The terms “antibody” or “immunoglobulin”, as used herein, have the same meaning, and will be used equally in the present invention. The term “antibody” as used herein refers to immunoglobulin molecules and immunologically active portions of immunoglobulin molecules, i.e., molecules that contain an antigen binding site that specifically binds an antigen. As such, the term antibody encompasses not only whole antibody molecules, but also antibody fragments or derivatives.

The term “binding affinity”, as used herein, refers to the strength of interaction between an antigen's epitope and an antibody's antigen binding site.

As used herein, a “promoter” is a specific nucleic acid sequence that is recognized by a DNA-dependent RNA polymerase (“transcriptase”) as a signal to bind to the nucleic acid and begin the transcription of RNA at a specific site.

The terms “modified sequence” and “modified genes” are used interchangeably herein to refer to a sequence that includes a deletion, insertion or interruption of naturally occurring nucleic acid sequence. In some preferred embodiments, the expression product of the modified sequence is a truncated protein (e.g., if the modification is a deletion or interruption of the sequence). In some particularly preferred embodiments, the truncated protein retains biological activity. In alternative embodiments, the expression product of the modified sequence is an elongated protein (e.g., modifications comprising an insertion into the nucleic acid sequence). In some embodiments, an insertion leads to a truncated protein (e.g., when the insertion results in the formation of a stop codon). Thus, an insertion may result in either a truncated protein or an elongated protein as an expression product.

As used herein, the terms “mutant sequence” and “mutant gene” are used interchangeably and refer to a sequence that has an alteration in at least one codon occurring in a host cell's wild-type sequence. The expression product of the mutant sequence is a protein with an altered amino acid sequence relative to the wild-type. The expression product may have an altered functional capacity (e.g., enhanced binding affinity).

The term “region” or “fragment” as used herein, refers to a portion of an amino acid sequence wherein said portion is smaller than the entire amino acid sequence. In some embodiments, refers to a portion of the receptor-binding domain (RBD) of the SARS-COV-2 with a sequence identity of at least about 90% to the amino acid sequence of the RBD. In some embodiments, refers to a portion of the spike protein outside the RBD of the SARS-COV-2 with a sequence identity of at least about 90% to the amino acid sequence of the spike protein outside the RBD.

The term “receptor-binding domain” or “RBD” refers to a protein in SARS-COV-2 S that bound strongly to human and bat angiotensin-converting enzyme 2 (ACE2) receptors.

The term “spike protein”, “S protein” or “S” refers to a large type I transmembrane protein ranging from 1,160 amino acids for avian infectious bronchitis virus (IBV) and up to 1,400 amino acids for feline coronavirus (FCoV). In addition, this protein is highly glycosylated as it contains 21 to 35 N-glycosylation sites. Spike proteins assemble into trimers on the virion surface to form the distinctive “corona”, or crown-like appearance. The ectodomain of all CoV spike proteins share the same organization in two domains: a N-terminal domain named S1 that is responsible for receptor binding and a C-terminal S2 domain responsible for fusion. CoV diversity is reflected in the variable spike proteins (S proteins), which have evolved into forms differing in their receptor interactions and their response to various environmental triggers of virus-cell membrane fusion. It's been reported that 2019-nCOV can infect the human respiratory epithelial cells through interaction with the human ACE2 receptor. Indeed, the recombinant Spike protein can bind with recombinant ACE2 protein.

The term “angiotensin converting enzyme 2” or “ACE2” refers to an enzyme attached to the cell membranes of cells in the lungs, arteries, heart, kidney, and intestines. ACE2 lowers blood pressure by catalysing the hydrolysis of angiotensin II (a vasoconstrictor peptide) into angiotensin (1-7) (a vasodilator). ACE2 counters the activity of the related angiotensin-converting enzyme (ACE) by reducing the amount of angiotensin-II and increasing Ang(1-7) making it a promising drug target for treating cardiovascular diseases. ACE2 also serves as the entry point into cells for some coronaviruses, including HCoV-NL63, SARS-COV, and SARS-COV-2. The human version of the enzyme is often referred to as hACE2.

The term “horseradish peroxidase” or “HRP” is used extensively in biochemistry applications. It is a metalloenzyme with many isoforms, of which the most studied type is C. It catalyzes the oxidation of various organic substrates by hydrogen peroxide.

As used herein, the term “N-terminal signal peptide” is a short peptide (usually 10-30 amino acids long) present at the N-terminus of the majority of newly synthesized proteins that are destined toward the secretory pathway. These proteins include those that reside either inside certain organelles (the endoplasmic reticulum, Golgi or endosomes), secreted from the cell, or inserted into most cellular membranes. Although most type I membrane-bound proteins have signal peptides, the majority of type II and multi-spanning membrane-bound proteins are targeted to the secretory pathway by their first transmembrane domain, which biochemically resembles a signal sequence except that it is not cleaved. They are a kind of target peptide.

As used herein, the term “purification tag” or “affinity tag” refers to a polypeptide used to purify proteins that simplifies purification and enables use of standard protocols. In the present invention, the purification tag is a polyhistidine tag of 4, 6, 7, 8, 9, 10, 11 or 12 histidine residues. Preferably, the histidine tag has 8 or 10 histidine residues.

As used herein, the term “linker” refers to a polypeptide comprising of 1-10 amino acids, preferably 3-6 amino acids. The amino acids of the linker may be selected from the group consisting of leucine (Leu, L), isoleucine (Ile, I), alanine (Ala, A), glycine (Gly, G), valine (Val, V), proline (Pro, P), lysine (Lys, K), arginine (Arg, R), Serine (Ser, S), asparagine (Asn, N), and glutamine (Gln, Q), tryptophan (Trp, W), methionine (Met, M), aspartic acid (Asp, D), cysteine (Cys, C), glutamic acid (Glu, E), histidine (His, H), phenylalanine (Phe, F), threonine (The, T), and tyrosine (Tyr, Y). In some preferred embodiments, the linker is a flexible linker that may consist of a sequence of consecutive amino acids that typically include at least one glycine and at least one serine. Exemplary flexible linkers include the amino acid sequences set forth in SEQ ID NO: 3 (GGGS), SEQ ID NO: 4 (GGGP), SEQ ID NO: 5 (GGSGG) or SEQ ID NO: 6 (GGSGGGGS), although the precise amino acid sequence of the linker is not particularly limiting. As used herein, the term “oligomerization tag” refers to a polypeptide for increasing assay avidity and sensitivity. In the present invention, the oligomerization tag are selected from a murine IgG1-Fc (CH2, CH3 only), a murine IgG1-Fc dimerization domain, a murine IgG-2a-Fc (CH2, CH3 only), a murine IgG-2a-Fc dimerization domain, a p53 tetramerization domain, a SARS-COV-2 nucleocapsid N-terminal domain and a SARS-COV-2 nucleocapsid C-terminal domain.

As used herein, the term “region in spike protein outside RBD” refers to a polypeptide comprising of 1-30 amino-acids of SARS-COV-2 which are not part of the RBD protein.

As used herein, the term “horseradish peroxidase binding domain” refers to an enzyme used in conjugates (molecules that have been joined genetically or chemically) to determine the presence of a molecular target.

As used herein, the term “tobacco etch virus cleavage site” or “TEV” refers to a highly site-specific cysteine protease that is found in the tags from fusion proteins. The optimal temperature for cleavage is 30° C.; also it can be used at temperature as low as 4° C. It is recommended that the cleavage for each fusion protein be optimized by varying the amount of recombinant viral TEV protease, reaction time, or incubation temperature. It can be removed by Ni²⁺ affinity resin. The optimum recognition site for this enzyme is the sequence Glu-Asn-Leu-Tyr-Phe-Gln-(Gly/Ser) [ENLYFQ(G/S)] and cleavage occurs between the Gln and Gly/Ser residues. The most commonly used sequence is ENLYFQG. The protease is used to cleave affinity tags from fusion proteins.

The term “diagnostic” or “diagnosed”, as used herein, means identifying the presence or nature of a pathologic condition or a patient susceptible to a disease. Diagnostic methods differ in their sensitivity and specificity. The “sensitivity” of a diagnostic assay is the percentage of diseased individuals who test positive (percent of “true positives”). Diseased individuals not detected by the assay are “false negatives”. Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

As used herein, the term “Biolayer interferometry (BLI)” is a label-free technology for measuring biomolecular interactions. It is an optical analytical technique that analyzes the interference pattern of white light reflected from two surfaces: a layer of immobilized protein on the biosensor tip, and an internal reference layer. Any change in the number of molecules bound to the biosensor tip causes a shift in the interference pattern that can be measured in real-time.

### I. FUSION PROTEINS

The present invention relates to a fusion protein comprising the SARS-COV-2 receptor binding domain (RBD) of the SARS-COV-2 spike protein or a fragment thereof, and a N-terminal signal peptide, and at least one of a polyhistidine tag, a linker, a oligomerization tag, a region in spike protein outside RBD, a horseradish peroxidase binding domain or a protease cleavage site.

The SARS-COV-2 full length Spike (FLS, GenBank MN908947.3), comprises two domains, namely S1 and S2, are responsible for the binding step. S1 contains the RBD, which directly binds to the peptidase domain (PD) of ACE2, whereas S2 is responsible for membrane fusion. When S1 binds to the host receptor ACE2, another cleavage site on S2 is exposed and is cleaved by host proteases, a process that is critical for viral infection. The S protein of SARS-COV-2 may also exploit ACE2 for host infection.

The fusion proteins of the present invention can be obtained by methods well-known to the skilled person. For example, said fusion proteins can be obtained recombinantly in bacteria, yeasts, fungi, or mammalian cells. In one embodiment, the fusion proteins of the present invention are produced in prokaryotic cells, such as Escherichia coli, but other prokaryotic cells can be used. In another embodiment, the fusion proteins of the present inventions are produced in human embryotic kidney (HEK) or Chinese hamster ovary (CHO) cells, but other eukaryotic cells can be used.

The fusion proteins of the present invention can be purified from the cells by methods well known to the skilled person. Said methods include, without limitation, filtration, conjugation, affinity chromatography, ion exchange chromatography, hydrophobic interaction chromatography, and size exclusion chromatography.

Regarding the signal peptides included in the fusion proteins of the present invention, these signal peptides could result in improved expression and/or secretion of the protein during recombinant production. Moreover, inclusion of different signal peptides can alter post translational modification (PTMs) and potentially the function of the protein. Therefore, it is non-obvious that the fusion proteins of the present invention can be produced or be functional. In one embodiment, said N-terminal signal peptide is selected from a spike endogenous signal peptide and a tissue plasminogen activator (tPa). Said N-terminal signal peptide has an amino acid sequence selected from SEQ ID NO:1 and SEQ ID NO:2.

As previously described, the use of polyhistidine tag simplifies purification and enables use of standard protocols in the production of fusion proteins. For example, the histidine (His) tag (also known as polyhistidine or polyHis) is known to be useful, for example, in the purification by Immobilized Metal Affinity Chromatography (IMAC). Other uses of the polyhistidine tag are also well-known by the skilled person and therefore the polyhistidine tag of the present invention is not limited to the purification functionality. In the present invention, said polyhistidine tag can be of 6, 8 or 10 histidine residues. It is important to evaluate the impact of a tag at both the N and C termini of the protein both to produce the protein but also for the functionality and aggregation states of the protein. The impact the location of the tag will have is non-obvious. Moreover, the utility of the tag in purification or any assay development is unknown. The inclusion of the TEV cleavage site was done with N-terminal tagging. If an N-terminally tagged construct were chosen, it would be possible to generate a tag free version. Additionally, the promiscuity of the TEV tag was utilized to support the possible production of a scar-free protein. Preferably said polyhistidine tag has an amino acid sequence selected from SEQ ID NO:7 and SEQ ID NO:8.

In another embodiment, oligomerization tags or domains have been included in the fusion proteins of the present invention which are selected from a murine IgG1-Fc (CH2, CH3 only), a murine IgG1-Fc dimerization domain, a murine IgG-2a-Fc (CH2, CH3 only), a murine IgG-2a-Fc dimerization domain, a p53 tetramerization domain, a SARS-COV-2 nucleocapsid N-terminal domain and a SARS-COV-2 nucleocapsid C-terminal domain. Said oligomerization tag has an amino acid sequence selected from SEQ ID NO:9, SEQ ID NO: 10, SEQ ID NO: 11, SEQ ID NO: 12, SEQ ID NO: 13, SEQ ID NO: 14 and SEQ ID NO: 15. The RBD molecular designs contain IgG1, IgG2aFc and p53 dimerization and tetramerization domains with the goal of increasing assay avidity and sensitivity.

Linkers can be also present in the fusion proteins of the present invention. In one embodiment, said linker can be a flexible linker. Flexible linkers are included when fusing domains of different proteins together. Most of these linkers are a combination of glycine and serine while in some cases proline was added to kink the protein. These flexible linkers may help to improve the tolerance for assembly of those domains, and are often a combination of glycine and serine. However, it is not obvious to the skilled person if the inclusion of the selected linkers would produce functional fusion proteins. In one embodiment, said linker is a flexible linker to add flexibility. Said linker has an amino acid sequence selected from SEQ ID NO:3, SEQ ID NO:4, SEQ ID NO:5 and SEQ ID NO:6.

The use of streptavidin binding domain (SBP) (SEQ ID NO: 17) to support assay development in either plate coating or conjugation of fluorophores or HRP tags for readout. The goal was to avoid labelling residues key to the protein interaction with hACE2 receptor or antibodies. A horseradish peroxidase (HRP) binding domain refers to an enzyme used in conjugates (molecules that have been joined genetically or chemically) to determine the presence of a molecular target. In some embodiments, said horseradish peroxidase binding domain has an amino acid sequence selected from SEQ ID NO: 18.

In some embodiments, said protease cleavage site is a tobacco etch virus cleavage site (TEV). Said protease cleavage site has an amino acid sequence selected from SEQ ID NO: 19.

In some embodiments, said receptor binding domain (RBD) of the SARS-COV-2 spike protein or a fragment thereof has an amino acid sequence of at least 90%, or at least 95% sequence identity with SEQ ID NO:20.

This invention also encompasses high affinity RBD mutations in specific RBD formats, in order to cover emergent SARS-COV-2 mutations that enhance binding to hACE2. Some of these novel protein designs harbor SARS-COV-2 mutations that emerged in nature (Pango lineage variants: B1.1.7, B. 1.351, B1.617.2, B.1.427 and P.1). In addition, molecular dynamic simulation and affinity maturation software from Schrodinger (Bio luminate) was used to predict the AA mutations in the primary sequence of RBD that would confer higher affinity to hACE2. Among those mutations we found that in silico, and in light to what has been described in the literature mutations V367F and G502D which increase expression of RBD and N501F, N501T and Q498Y.

### II. EXEMPLARY FUSION PROTEINS

In some embodiments, said fusion protein has an amino acid sequence of at least 90%, or at least 95% sequence identity with SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO: 43, SEQ ID NO: 44, SEQ ID NO: 45, SEQ ID NO:46, SEQ ID NO:47, SEQ ID NO: 48, SEQ ID NO: 49, SEQ ID NO:50, SEQ ID NO:51, SEQ ID NO:52, SEQ ID NO:53, SEQ ID NO:54, SEQ ID NO:55, SEQ ID NO:56, or SEQ ID NO:57.

The present inventors also designed embodiments where the RBD with N proteins are fused together, as well as RBD and HRP with the goal of improving assay sensitivity during the acute phase of infection, as N protein is detected early during the infection.

In some embodiments, the invention also embodies high affinity RBD mutations that enhance binding to human ACE2. The present inventors used molecular dynamic simulation and affinity maturation software from Schrodinger (Bio luminate) to predict the AA mutations in the primary sequence of RBD that would confer higher affinity to hACE2. Among those mutations are V367F and G502D which increases expression of RBD and N501F, N501T and Q498Y. In some embodiments, said SARS-COV-2 RBD protein comprises a mutation in one or more of the following positions: G404, A475, T478, N481, G485, F490, Q493, G496, Q498, N501, or V503.

### III. NUCLEIC ACIDS, CLONING CELLS, AND EXPRESSION CELLS

The present invention also relates to nucleic acids comprising a nucleotide sequence encoding the fusion proteins described herein. The nucleic acid may be DNA or RNA. DNA comprising a nucleotide sequence encoding a fusion protein described herein typically comprises a promoter that is operably-linked to the nucleotide sequence. The promoter is preferably capable of driving constitutive or inducible expression of the nucleotide sequence in an expression cell of interest. Said nucleic acid may also comprise a selectable marker useful to select the cell containing said nucleic acid of interest. Useful selectable markers are well known by the skilled person. The precise nucleotide sequence of the nucleic acid is not particularly limiting so long as the nucleotide sequence encodes a fusion protein described herein. Codons may be selected, for example, to match the codon bias of an expression cell of interest (e.g., a mammalian cell such as a human cell) and/or for convenience during cloning. DNA may be a plasmid, for example, which may comprise an origin of replication (e.g., for replication of the plasmid in a prokaryotic cell).

In one embodiment described herein, the present invention refers to a nucleic acid comprising a nucleotide sequence encoding the fusion protein, a promoter operably linked to the nucleotide sequence and a selectable marker.

Various aspects of the present invention also relate to a cell comprising a nucleic acid comprising a nucleotide sequence that encodes a fusion protein as described herein. The cell may be an expression cell or a cloning cell. Nucleic acids are typically cloned in E. coli, although other cloning cells may be used.

If the cell is an expression cell, the nucleic acid is optionally a nucleic acid of a chromosome, i.e., wherein the nucleotide sequence is integrated into the chromosome, although then nucleic acid may be present in an expression cell, for example, as extrachromosomal DNA or vectors, such as plasmids, cosmids, phages, etc. The format of the vector should not be considered limiting.

In one embodiment described herein, the cell is typically an expression cell. The nature of the expression cell is not particularly limiting. Expression cells which may be used are prokaryotic cells such as E. coli and Bacillus spp. and eukaryotic cells such as yeast cells (e.g. S. cerevisiae, S. pombe, P. pastoris, K lactis, H polymorpha), insect cells (e.g. Sf9), fungal, plant cells or mammalian cells. Mammalian expression cells may allow for favorable folding, post-translational modifications, and/or secretion of a fusion protein, although other eukaryotic cells or prokaryotic cells may be used as expression cells. Exemplary expression cells include TunaCHO, ExpiCHO, Expi293, BHK, NS0, Sp2/0, COS, C127, HEK, HT-1080, PER.C6, HeLa, and Jurkat cells. The cell may also be selected for integration of a vector, more preferably for integration of a plasmid DNA.

The fusion proteins of the present invention can be produced by appropriate transfection strategy of the nucleic acids comprising a nucleotide sequence that encodes the fusion proteins into mammalian cells. The skilled person is aware of the different techniques available for transfection of nucleic acids into the cell line of choice (lipofection, electroporation, etc). Thus, the choice of the mammalian cell line and transfection strategy should not be considered limiting. The cell line could be further selected for integration of the plasmid DNA.

Various aspects of the present invention also relate to a cell comprising the fusion proteins described herein.

### IV. COMPOSITIONS AND METHODS RELATED TO ASSAYS

Various aspects of the present invention relate to compositions comprising a fusion protein as described herein. In some embodiments, the composition may comprise a pharmaceutically-acceptable carrier and/or a pharmaceutically-acceptable excipient. The composition may be, for example, a vaccine.

Various embodiments of the present invention relate to a method of treating or preventing a SARS-COV-2 infection in a human patient comprising administering to the patient a composition comprising a fusion protein as described herein. The term “preventing” as used herein refers to prophylaxis, which includes the administration of a composition to a patient to reduce the likelihood that the patient will become infected with SARS-COV-2 relative to an otherwise similar patient who does not receive the composition. The term preventing also includes the administration of a composition to a group of patients to reduce the number of patients in the group who become infected with SARS-COV-2 relative to an otherwise similar group of patients who do not receive the composition.

Various embodiments of the invention relate to a method of treating or preventing a SARS-COV-2 infection in a human patient comprising administering to the patient a vaccine according to the embodiments described herein.

A patient may be infected with SARS-COV-2, a patient may have been exposed to SARS-COV-2, or a patient may present with an elevated risk for exposure to and/or infection with SARS-COV-2.

In one embodiment described herein, the composition comprises the fusion protein of the present invention and a solid support.

In other embodiment, the composition comprises the fusion protein of the present invention and a solid support, wherein the fusion protein is covalently or non-covalently bound to the solid support. The term “non-covalently bound,” as used herein, refers to specific binding such as between an antibody and its antigen, a ligand and its receptor, or an enzyme and its substrate, exemplified, for example, by the interaction between streptavidin binding protein and streptavidin or an antibody and its antigen.

In other embodiment, the composition comprises the fusion protein of the present invention and a solid support, wherein the fusion protein is directly or indirectly bound to a solid support. The term “direct” binding, as used herein, refers to the direct conjugation of a molecule to a solid support, e.g., a gold-thiol interaction that binds a cysteine thiol of a fusion protein to a gold surface. The term “indirect” binding, as used herein, includes the specific binding of a fusion protein to another molecule that is directly bound to a solid support, e.g., a fusion protein may bind an antibody that is directly bound to a solid support thereby indirectly binding the fusion protein to the solid support. The term “indirect” binding is independent of the number of molecules between the fusion protein and the solid support so long as (a) each interaction between the daisy chain of molecules is a specific or covalent interaction and (b) a terminal molecule of the daisy chain is directly bound to the solid support.

A solid support may comprise a particle, a bead, a membrane, a surface, a polypeptide chip, a microtiter plate, or the solid-phase of a chromatography column.

A composition may comprise a plurality of beads or particles, wherein each bead or particle of the plurality of beads or particles are directly or indirectly bound to at least one fusion protein as described herein. A composition may comprise a plurality of beads or particles, wherein each bead or particle of the plurality of beads or particles are covalently or non-covalently bound to at least one fusion protein as described herein.

Various aspects of the embodiments relate to a kit for detecting the presence of antibodies against the fusion protein of the present invention, and/or fragment thereof in a sample, said kit comprising a fusion protein and a solid support or composition as described herein.

The compositions and kits described herewith can be either for use in an assay or in compositions that are generated during the performance of an assay. Various aspects of the invention relate to a diagnostic medical device comprising a composition as described herein.

Various aspects of the invention relate to assays for detection of anti-SARS-COV-2 antibodies.

An assay may be an assay for measuring the relative binding affinity of the fusion protein of the present invention to anti-RBD, fragment anti-RBD and/or fragment anti-RBD in a sample (e.g., relative to one or more control samples or standards). An assay may be an assay for measuring the relative binding affinity of the fusion protein of the present invention to any anti-RBD (e.g., relative to one or more control samples or standards).

Assays typically feature a solid support that either allows for measurement, such as by turbidimetry, nephelometry, UV/Vis/IR spectroscopy (e.g., absorption, transmission), fluorescence or phosphorescence spectroscopy, or surface plasmon resonance, or aids in the separation of components that directly or indirectly bind the solid support from components that do not directly or indirectly bind the solid support, or both. For example, an assay may include a composition comprising particles or beads and/or that aid in the mechanical separation of components that directly or indirectly bind the particles or beads.

Other exemplary assays that may include the fusion protein or the composition of the present invention includes but it is not limited to ELISA, lateral flow, single molecule counting (SMC), viscoelastic tests such as Sonoclot, gel technologies, fluorescence assay and other point-of-care testing using any of these techniques.

The fusion proteins of the present invention will be further illustrated by the following non-limiting examples.

### EXAMPLES

### Example 1: Expression and Purification of pxENB14-RBD and pxENB17-RBD Proteins of the Present Invention

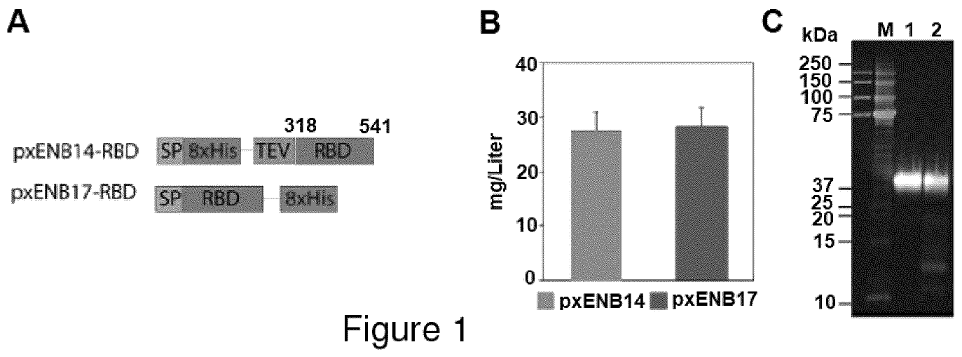

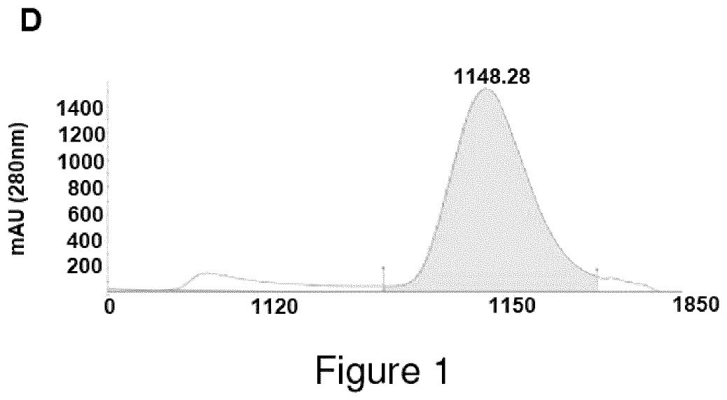

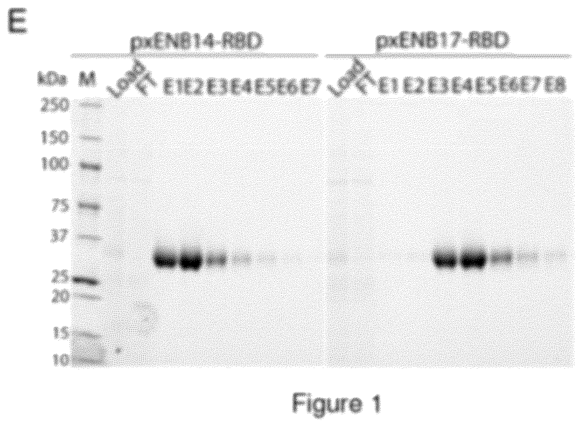

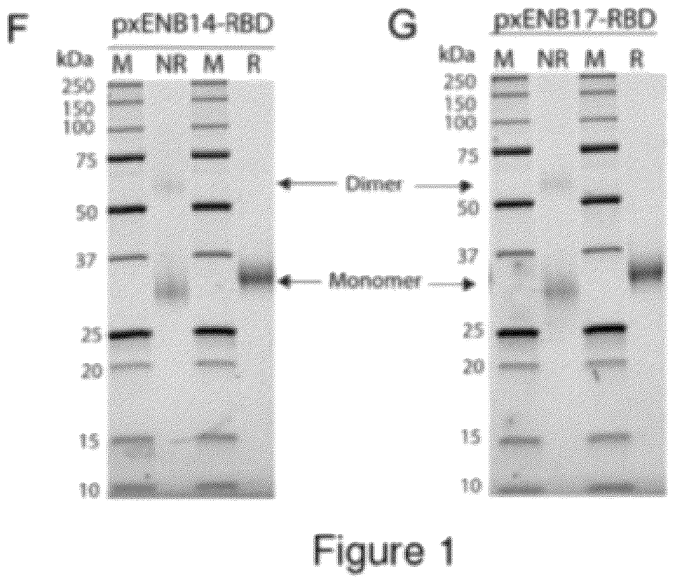

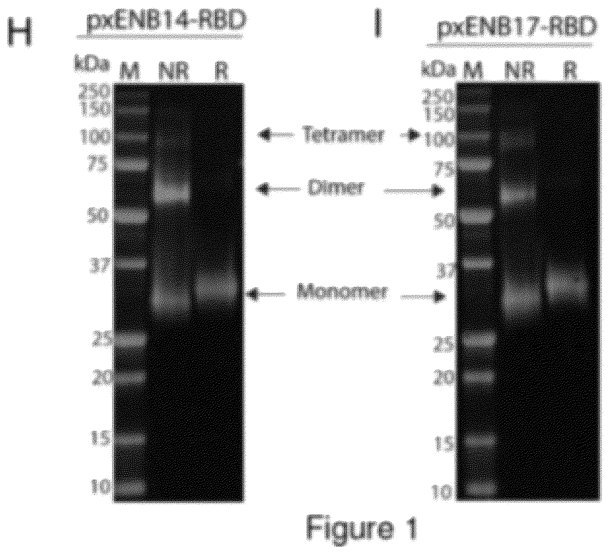

The RBD proteins were produced in Expi293 cells and affinity purified from the supernatant. The affinity purification was carried out according to IMAC standard protocols that include imidazole washes and elution. After spin concentration and buffer exchange, the proteins were subjected to functional evaluation by SDS-PAGE Western-blot under reducing and non-reducing conditions. FIG. 1 shows experimental data for two molecular designs, final purified samples characterized by SDS-PAGE.

Evaluation of pxENB14-RBD and pxENB17-RBD proteins by SDS-PAGE Western-blot revealed existence of RBD monomers, dimers and tetramers. This data was corroborated by SECMALS. Both proteins were recognized by rabbit polyclonal antibodies on a Western blot, demonstrating bioactivity. Intact mass analysis was performed using N— and O, D-, glycosylation and reducing conditions (Table 1). Both pxENB14-RBD and pxENB17-RBD showed the shame MW shift suggesting the existence of a non-identified PTM by intact mass spectrometry analysis.

TABLE 1
Final Molecular Weight measured by Intact Mass Spectrometry

| | Theoretical | Measured | |
|---|---|---|---|
| Construct | MW (Da) | MW (Da) | Comments |
| pxENB14-RBD | 27248.63 | 27473.4 | Δ MW = 224.77 Da |
| pxENB17-RBD | 26453.77 | 26678.5 | Δ MW = 224.73 Da |

### Example 2: Evaluation of RBD-hACE2 Interaction

The diversity of SARS-COV-2 pandemic RBD sequences remains low. However, a subset of mutations has been observed, with 10 particular mutants appearing to be under high positive selection pressure to spread across the world. According to some studies, three RBD mutants emerged in Wuhan, Shenzhen, Hong Kong and France and these mutants showed higher affinity to the ACE2 receptor when in comparison with to the prototype Wuhan-Hu-1 strain. Two mutations (F342L, R408I) showed similar affinity to ACE2 as the original Wuhan strain but four mutations were identified (N354D, D364Y, V367F, W436R) (Ou, J. et al. 2020, bioRxiv, doi: https://doi.org/10.1101/2020.03.15.991844).

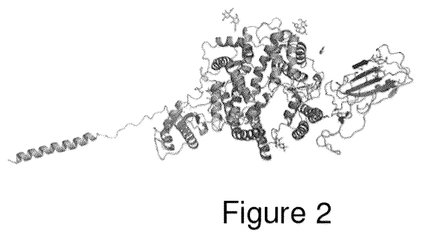

In light of the emergent RBD mutations, protein modelling was performed with residue scanning and affinity maturation of a structure of SARS-COV-2 receptor-binding domain in complex with the human ACE2 receptor. These studies were performed using Schrodinger's BioLuminate Software and were focused on the RBD-ACE2 interaction (FIG. 2).

### Example 3: Evaluation of Receptor Binding Domain Mutations

The goal of this study was to identify novel and potential emergent mutations that could result in stronger binding to ACE2. The results from the study are summarized in Table 2. These mutations can be utilized individually or in combination and the number of mutations is not limiting for any of the designs proposed in the present invention.

In order to find high affinity RBD mutations that enhance binding to human ACE2, the present inventors used molecular dynamic simulation and affinity maturation software from Schrodinger (Bio luminate) to predict the AA mutations in the primary sequence of RBD that would confer higher affinity to hACE2. Among those mutations are V367F and G502D, which increased expression of RBD and N501F, N501T and Q498Y.

TABLE 2
RBD mutants identified by residue scan and affinity maturation.

| Position | Identified mutation |
|---|---|
| G404 | Affinity Maturation: R, S, V |
| A475 | Affinity Maturation: R, M |
| T478 | Affinity Maturation: K |
| N481 | Affinity Maturation: K, V, W |
| G485 | Affinity Maturation: R |
| F490 | Affinity Maturation: R, Q, T |
| Q493 | Affinity Maturation: R, M, K |
| G496 | Affinity Maturation: R |
| Q498 | Affinity Maturation: R, M, Y |
| N501 | Affinity Maturation: H |
| V503 | Affinity Maturation: W |

### Example 4: Confirmation of Functionality of pxENB14-RBD and pxENB17-RBD Proteins of the Present Invention

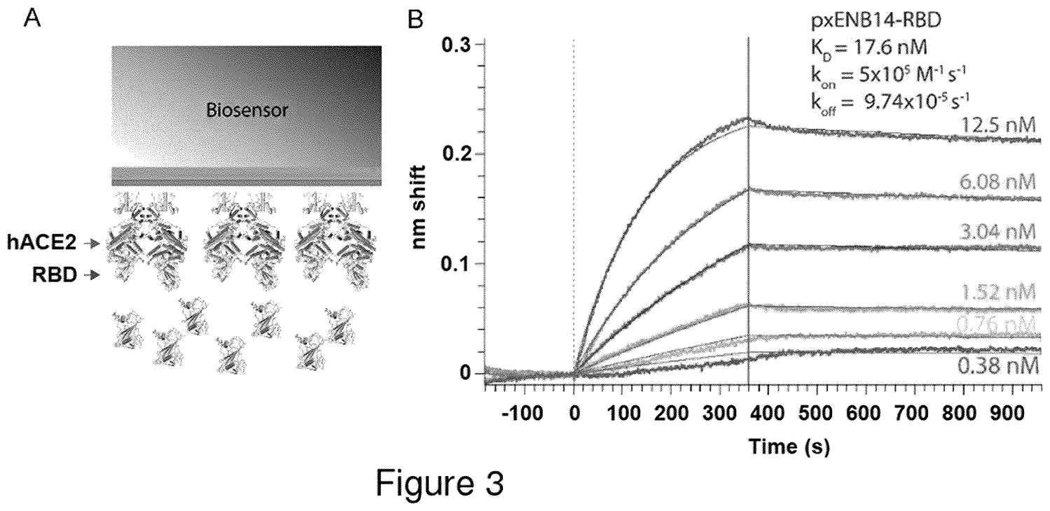

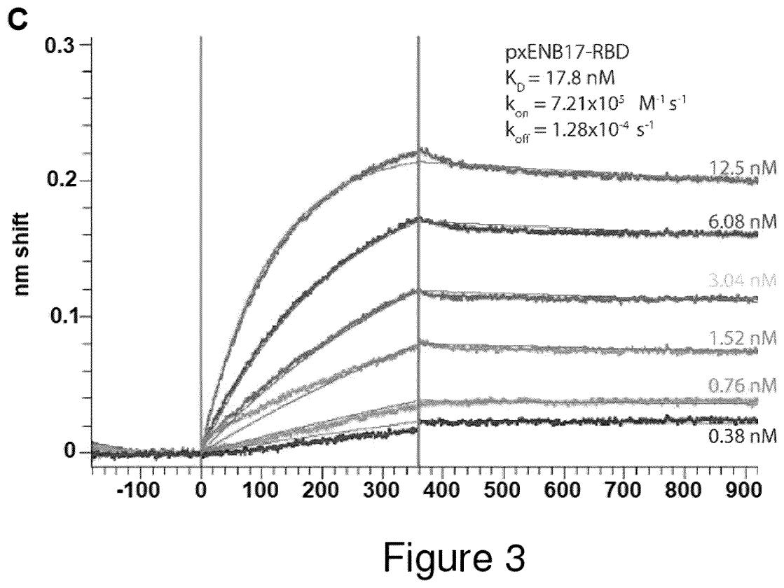

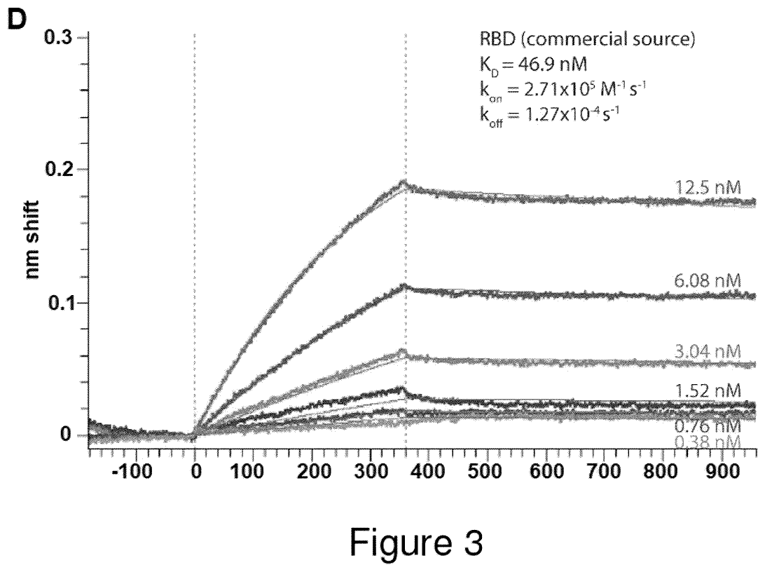

The functionality of pxENB14-RBD and pxENB17-RBD was evaluated by BLI. Briefly, biotinylated hACE2 was immobilized on the surface of a streptavidin biosensor and incubated with RBD proteins at concentrations ranging from 12.5 to 0.38 nM (FIG. 3). Based on KD values, pxENB14-RBD and pXENB17-RBD show superior affinity compared to RBD from a commercial source; suggesting that RBD proteins are more potent.

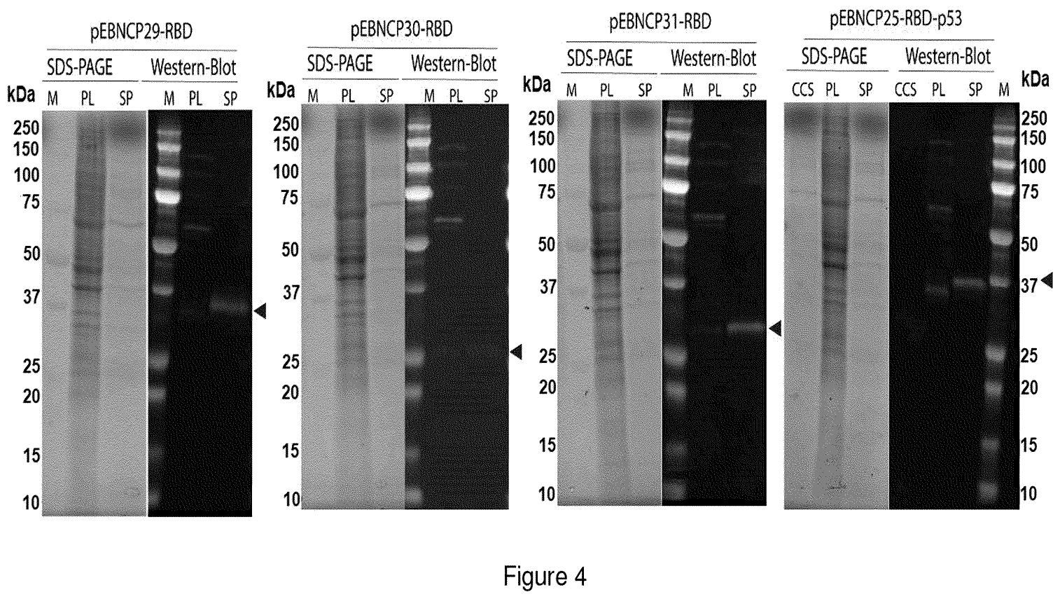

The inventors evaluated the expression of a subset of RBD truncations and fusions in Expi293. The RBD truncations and multimeric versions were produced in Expi293 cells (FIG. 4). Expression evaluation was performed by SDS-PAGE and Western-blot under reducing conditions. All constructs expressed and secreted the protein to the cell culture supernatant.

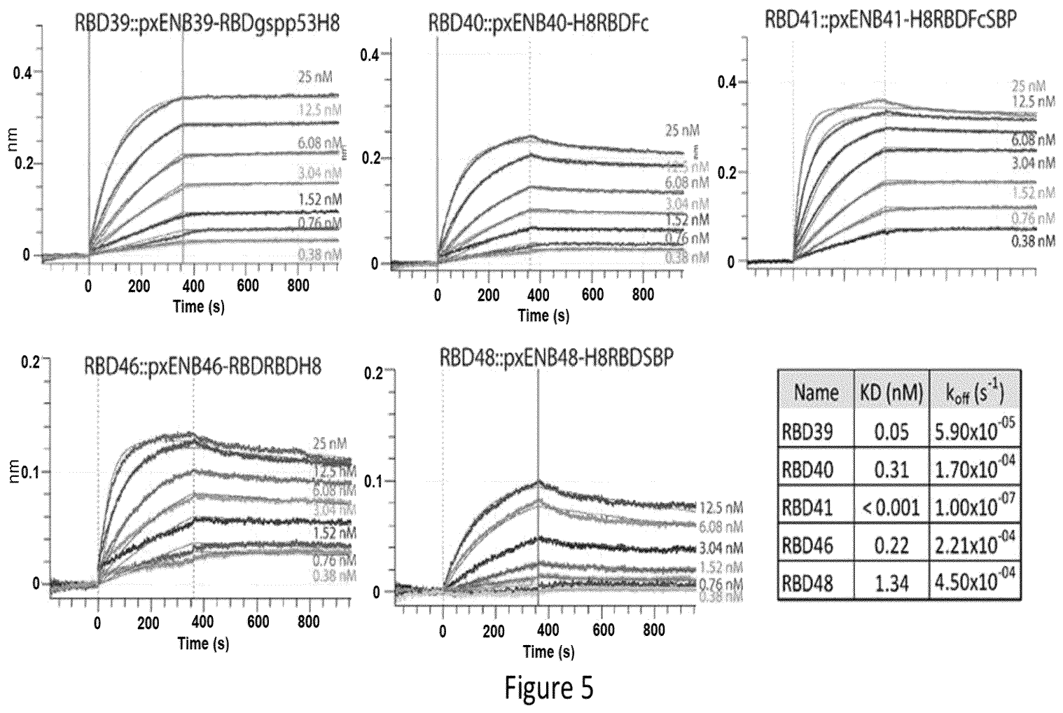

In addition, multimeric RBD proteins were incubated at protein concentrations ranging from 25 to 0.38 nM and tested by binding to biotinylated hACE2 immobilized on the surface of streptavidin biosensors, similarly to what has been described in FIG. 3. All proteins tested, except RBD41, show tighter binding to rhACE2 than pxENB14, as observed by the values for the rates of dissociation (koff), see FIG. 5.

Binding curves of immobilized hACE2 with SARS-COV-2 multimeric RBD proteins in FIG. 5 show that addition of multimeric domains increased avidity and has a positive effect in the k_{off} rate when compared to pxENB14RBD, except for RBD41. All proteins show rates of dissociation (k_{off}) lower than pxENB14RBD, suggesting tighter binding to rhACE2. Data is shown in different color lines depending on analyte concentration, and the data was best fitted to a 1:1 binding model as shown by the red line.

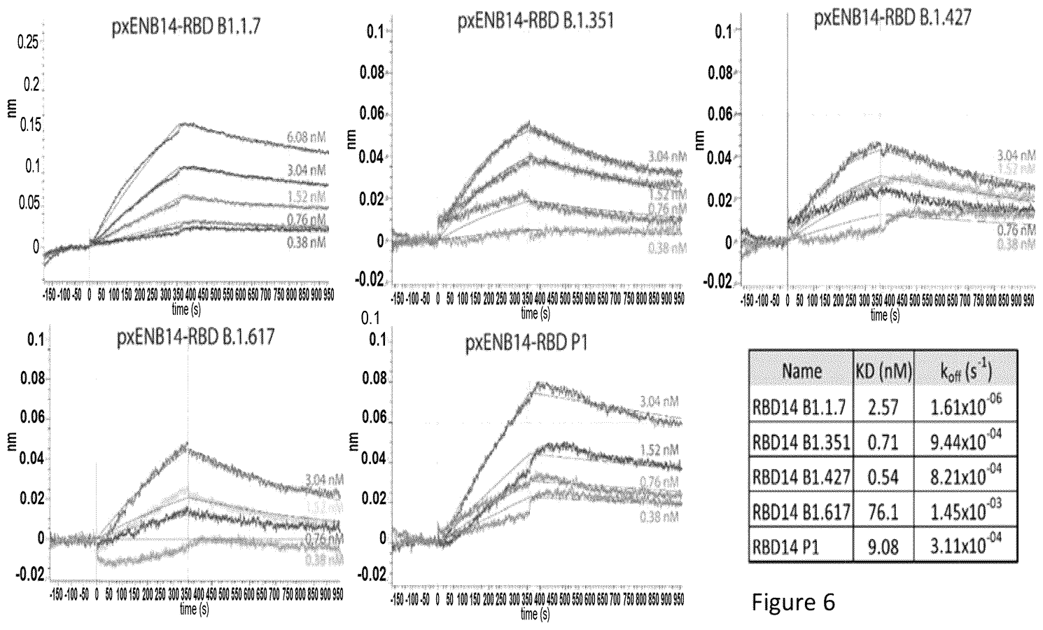

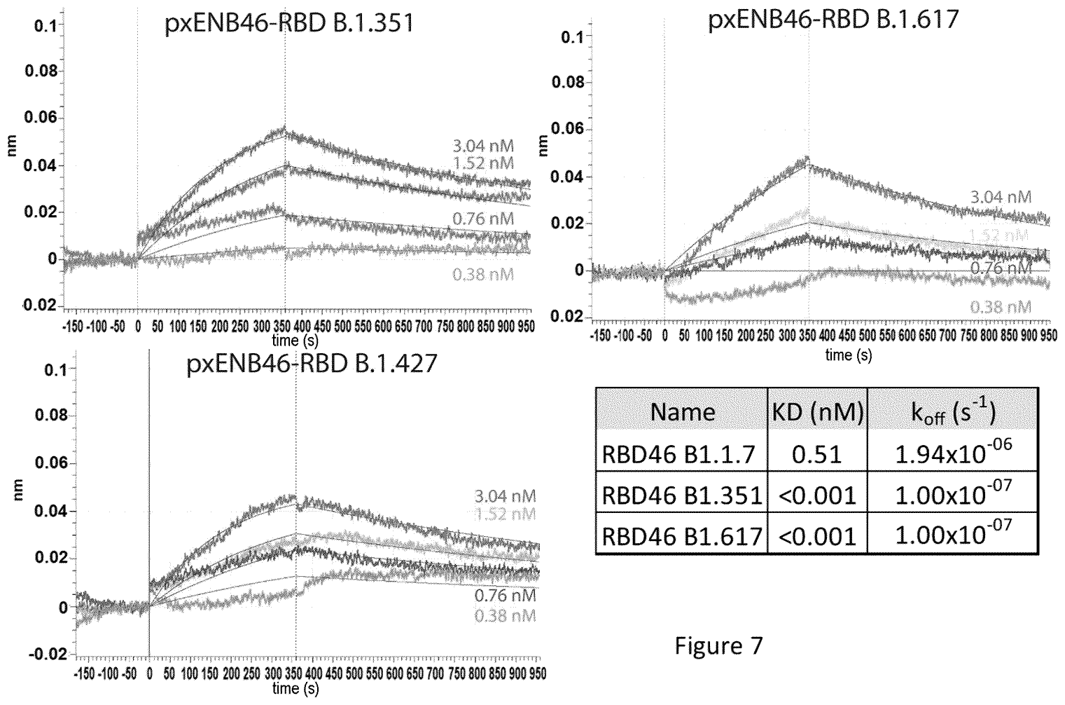

Functionality of the RBD mutant proteins by BLI based on pxENB14RBD (FIG. 6) and pxENB46RBD (FIG. 7).

FIG. 6 shows binding curves of immobilized hACE2 with SARS-COV-2 pxENB14RBD mutants (Pango lineages) that described current SARS-COV-2 variants.

Mutants pxENBRBD14-B1.617 (SEQ ID NO:52) shows a particular low affinity to the rhACE2 receptor, as seen by the increase observed in the affinity based on K_{D} values from 17 nM to 76.1 nM. All RBD mutants, except pxENB-RBD14 B1.617 (SEQ ID NO:52) show a lower rate of dissociation than pxENB14RBD, suggesting that these bind to the rhACE2 stronger than the original protein.

FIG. 7 shows the binding curves of immobilized hACE2 with SARS-COV-2 pxENB46RBD mutants (Pango lineages) that described current SARS-COV-2 variants.

SEQUENCES

| SEQ ID | | |
|---|---|---|
| NOs | Sequence (5′ to 3′) | Comments |
| SEQ ID | MFVFLVLLPLVSSQ | SARS-Cov-2 spike |
| NO: 1 | | protein Endogenous |
| | | signal peptide |
| SEQ ID | MDAMKRGLCCVLLLCGAVFVSPS | Tissue plasminogen |
| NO: 2 | | activator signal |
| | | peptide |
| SEQ ID | GGGS | Flexible linker |
| NO: 3 | | |
| SEQ ID | GGGP | Flexible linker |
| NO: 4 | | |
| SEQ ID | GGSGG | Flexible linker |
| NO: 5 | | |
| SEQ ID | GGSGGGGS | Flexible linker |
| NO: 6 | | |
| SEQ ID | HHHHHHHH | His tag (8x) |
| NO: 7 | | |
| SEQ ID | HHHHHHHHHH | His tag (10x) |
| NO: 8 | | |
| SEQ ID | VPEVSSVFIFPPKPKDVLTITLTPKVTCVVVDISKDDPEVQ | Murine IgG1-Fc |
| NO: 9 | FSWFVDDVEVHTAQTQPREEQFNSTFRSVSELPIMHQD | (CH2, CH3 only) tag |
| | WLNGKEFKCRVNSAAFPAPIEKTISKTKGRPKAPQVYTIP | (without hinge) |
| | PPKEQMAKDKVSLTCMITDFFPEDITVEWQWNGQPAEN | |
| | YKNTQPIMDTDGSYFVYSKLNVQKSNWEAGNTFTCSVL | |
| | HEGLHNHHTEKSLSHSPGI | |
| SEQ ID | VPRDCGCKPCICTVPEVSSVFIFPPKPKDVLTITLTPKVT | Murine IgG1-Fc |
| NO: 10 | CVVVDISKDDPEVQFSWFVDDVEVHTAQTQPREEQFNS | Dimerization domain |
| | TFRSVSELPIMHQDWLNGKEFKCRVNSAAFPAPIEKTISK | |
| | TKGRPKAPQVYTIPPPKEQMAKDKVSLTCMITDFFPEDIT | |
| | VEWQWNGQPAENYKNTQPIMDTDGSYFVYSKLNVQKS | |
| | NWEAGNTFTCSVLHEGLHNHHTEKSLSHSPGI | |
| SEQ ID | PSVFIFPPKIKDVLMISLSPIVTCVVVDVSEDDPDVQISWF | Murine IgG-2a-Fc |
| NO: 11 | VNNVEVHTAQTQTHREDYNSTLRVVSALPIQHQDWMSG | (CH2, CH3 only) tag |
| | KEFKCKVNNKDLPAPIERTISKPKGSVRAPQVYVLPPPE | (without hinge) |
| | EEMTKKQVTLTCMVTDFMPEDIYVEWTNNGKTELNYKN | |
| | TEPVLDSDGSYFMYSKLRVEKKNWVERNSYSCSVVHE | |
| | GLHNHHTTKSFSRTPGK | |
| SEQ ID | PRGPTIKPCPPCKCPAPNLLGGPSVFIFPPKIKDVLMISLS | Murine IgG2a-Fc |
| NO: 12 | PIVTCVVVDVSEDDPDVQISWFVNNVEVHTAQTQTHRE | Dimerization domain |
| | DYNSTLRVVSALPIQHQDWMSGKEFKCKVNNKDLPAPIE | |
| | RTISKPKGSVRAPQVYVLPPPEEEMTKKQVTLTCMVTDF | |
| | MPEDIYVEWTNNGKTELNYKNTEPVLDSDGSYFMYSKL | |
| | RVEKKNWVERNSYSCSVVHEGLHNHHTTKSFSRTPGK | |
| SEQ ID | KPLDGEYFTLQIRGRERFEMFRELNEALELKDAQAGKEP | p53 Tetramerization |
| NO: 13 | G | domain |
| SEQ ID | ASWFTALTQHGKEDLKFPRGQGVPINTNSSPDDQIGYY | SARS-CoV-2 |
| NO: 14 | RRATRRIRGGDGKMKDLSPRWYFYYLGTGPEAGLPYG | nucleocapsid N- |
| | ANKDGIIWVATEGALNTPKDHIGTRNPANNAAIVLQLPQG | terminal domain |
| | TTLPKGFYA | |
| SEQ ID | AEASKKNVTQAFGRRGPEQTQGNFGDQELIRQGTDYK | SARS-CoV-2 |
| NO: 15 | HWPQIAQFAPSASAFFGMSRIGMEVTPSGTWLTYTGAIK | nucleocapsid C- |
| | LDDKDPNFKDQVILLNKHIDAYKTF | terminal domain |
| SEQ ID | VPRDCGCKPCICT | Murine IgG1-Fc |
| NO: 16 | | Hinge Domain |
| SEQ ID | MDEKTTGWRGGHVVEGLAGELEQLRARLEHHPQGQRE | Streptavidin binding |
| NO: 17 | P | peptide tag |
| SEQ ID | QLTPTFYDNSCPNVSNIVRDTIVNELRSDPRIAASILRLHF | HRP enzyme |
| NO: 18 | HDCFVNGCDASILLDNTTSFRTEKDAFGNANSARGFPVI | |
| | DRMKAAVESACPRTVSCADLLTIAAQQSVTLAGGPSWR | |
| | VPLGRRDSLQAFLDLANANLPAPFFTLPQLKDSFRNVGL | |
| | NRSSDLVALSGGHTFGKNQCRFIMDRLYNFSNTGLPDP | |
| | TLNTTYLQTLRGLCPLNGNLSALVDFDLRTPTIFDNKYYV | |
| | NLEEQKGLIQSDQELFSSPNATDTIPLVRSFANSTQTFFN | |
| | AFVEAMDRMGNITPLTGTQGQIRLNCRVVNSNS | |
| SEQ ID | ENLYFQ | TEV Cleavage site |
| NO: 19 | | |
| SEQ ID | RVQPTESIVRFPNITNLCPFGEVFNATRFASVYAWNRKRI | RBD |
| NO: 20 | SNCVADYSVLYNSASFSTFKCYGVSPTKLNDLCFTNVYA | |
| | DSFVIRGDEVRQIAPGQTGKIADYNYKLPDDFTGCVIAW | |
| | NSNNLDSKVGGNYNYLYRLFRKSNLKPFERDISTEIYQA | |
| | GSTPCNGVEGFNCYFPLQSYGFQPTNGVGYQPYRVVV | |
| | LSFELLHAPATVCGPKKSTNLVKNKCVNF | |
| SEQ ID | MFVFLVLLPLVSSQHHHHHHHHGGGSENLYFQRVQPTE | pxENB14-RBD |
| NO: 21 | SIVRFPNITNLCPFGEVFNATRFASVYAWNRKRISNCVA | |
| | DYSVLYNSASFSTFKCYGVSPTKLNDLCFTNVYADSFVI | |
| | RGDEVRQIAPGQTGKIADYNYKLPDDFTGCVIAWNSNNL | |
| | DSKVGGNYNYLYRLFRKSNLKPFERDISTEIYQAGSTPC | |
| | NGVEGFNCYFPLQSYGFQPTNGVGYQPYRVVVLSFELL | |
| | HAPATVCGPKKSTNLVKNKCVNF | |
| SEQ ID | MFVFLVLLPLVSSQRVQPTESIVRFPNITNLCPFGEVFNA | pxENB17-RBD |
| NO: 22 | TRFASVYAWNRKRISNCVADYSVLYNSASFSTFKCYGV | |
| | SPTKLNDLCFTNVYADSFVIRGDEVRQIAPGQTGKIADY | |
| | NYKLPDDFTGCVIAWNSNNLDSKVGGNYNYLYRLFRKS | |
| | NLKPFERDISTEIYQAGSTPCNGVEGFNCYFPLQSYGFQ | |
| | PTNGVGYQPYRVVVLSFELLHAPATVCGPKKSTNLVKN | |
| | KCVNFGGGSHHHHHHHH | |
| SEQ ID | MDAMKRGLCCVLLLCGAVFVSPSHHHHHHHHGGGSEN | pxENB15-RBD |
| NO: 23 | LYFQRVQPTESIVRFPNITNLCPFGEVFNATRFASVYAW | |
| | NRKRISNCVADYSVLYNSASFSTFKCYGVSPTKLNDLCF | |
| | TNVYADSFVIRGDEVRQIAPGQTGKIADYNYKLPDDFTG | |
| | CVIAWNSNNLDSKVGGNYNYLYRLFRKSNLKPFERDIST | |
| | EIYQAGSTPCNGVEGFNCYFPLQSYGFQPTNGVGYQPY | |
| | RVVVLSFELLHAPATVCGPKKSTNLVKNKCVNF | |
| SEQ ID | MDAMKRGLCCVLLLCGAVFVSPSRVQPTESIVRFPNITN | pxENB18-RBD |
| NO: 24 | LCPFGEVFNATRFASVYAWNRKRISNCVADYSVLYNSA | |
| | SFSTFKCYGVSPTKLNDLCFTNVYADSFVIRGDEVRQIA | |
| | PGQTGKIADYNYKLPDDFTGCVIAWNSNNLDSKVGGNY | |
| | NYLYRLFRKSNLKPFERDISTEIYQAGSTPCNGVEGFNC | |
| | YFPLQSYGFQPTNGVGYQPYRVVVLSFELLHAPATVCG | |
| | PKKSTNLVKNKCVNFGGGSHHHHHHHH | |
| SEQ ID | MDAMKRGLCCVLLLCGAVFVSPSRVQPTESIVRFPNITN | pxEBNCP21-RBD |
| NO: 25 | LCPFGEVFNATRFASVYAWNRKRISNCVADYSVLYNSA | |
| | SFSTFKCYGVSPTKLNDLCFTNVYADSFVIRGDEVRQIA | |
| | PGQTGKIADYNYKLPDDFTGCVIAWNSNNLDSKVGGNY | |
| | NYLYRLFRKSNLKPFERDISTEIYQAGSTPCNGVEGFNC | |
| | YFPLQSYGFQPTNGVGYQPYRVVVLSFELLHAPATVCG | |
| | PKKSTNLVKNKCVNFVPRDCGCKPCICTVPEVSSVFIFP | |
| | PKPKDVLTITLTPKVTCVVVDISKDDPEVQFSWFVDDVE | |
| | VHTAQTQPREEQFNSTFRSVSELPIMHQDWLNGKEFKC | |
| | RVNSAAFPAPIEKTISKTKGRPKAPQVYTIPPPKEQMAKD | |
| | KVSLTCMITDFFPEDITVEWQWNGQPAENYKNTQPIMDT | |
| | DGSYFVYSKLNVQKSNWEAGNTFTCSVLHEGLHNHHTE | |
| | KSLSHSPGI | |
| SEQ ID | MDAMKRGLCCVLLLCGAVFVSPSRVQPTESIVRFPNITN | pxEBNCP22-RBD |
| NO: 26 | LCPFGEVFNATRFASVYAWNRKRISNCVADYSVLYNSA | |
| | SFSTFKCYGVSPTKLNDLCFTNVYADSFVIRGDEVRQIA | |
| | PGQTGKIADYNYKLPDDFTGCVIAWNSNNLDSKVGGNY | |
| | NYLYRLFRKSNLKPFERDISTEIYQAGSTPCNGVEGFNC | |
| | YFPLQSYGFQPTNGVGYQPYRVVVLSFELLHAPATVCG | |
| | PKKSTNLVKNKCVNFPRGPTIKPCPPCKCPAPNLLGGPS | |
| | VFIFPPKIKDVLMISLSPIVTCVVVDVSEDDPDVQISWFVN | |
| | NVEVHTAQTQTHREDYNSTLRVVSALPIQHQDWMSGKE | |
| | FKCKVNNKDLPAPIERTISKPKGSVRAPQVYVLPPPEEE | |
| | MTKKQVTLTCMVTDFMPEDIYVEWTNNGKTELNYKNTE | |
| | PVLDSDGSYFMYSKLRVEKKNWVERNSYSCSVVHEGL | |
| | HNHHTTKSFSRTPGK | |
| SEQ ID | MDAMKRGLCCVLLLCGAVFVSPSRVQPTESIVRFPNITN | pxEBNCP23-RBD |
| NO: 27 | LCPFGEVFNATRFASVYAWNRKRISNCVADYSVLYNSA | |
| | SFSTFKCYGVSPTKLNDLCFTNVYADSFVIRGDEVRQIA | |
| | PGQTGKIADYNYKLPDDFTGCVIAWNSNNLDSKVGGNY | |
| | NYLYRLFRKSNLKPFERDISTEIYQAGSTPCNGVEGFNC | |
| | YFPLQSYGFQPTNGVGYQPYRVVVLSFELLHAPATVCG | |
| | PKKSTNLVKNKCVNFGGGPVPEVSSVFIFPPKPKDVLTIT | |
| | LTPKVTCVVVDISKDDPEVQFSWFVDDVEVHTAQTQPR | |
| | EEQFNSTFRSVSELPIMHQDWLNGKEFKCRVNSAAFPA | |
| | PIEKTISKTKGRPKAPQVYTIPPPKEQMAKDKVSLTCMIT | |
| | DFFPEDITVEWQWNGQPAENYKNTQPIMDTDGSYFVYS | |
| | KLNVQKSNWEAGNTFTCSVLHEGLHNHHTEKSLSHSPG | |
| | I | |
| SEQ ID | MDAMKRGLCCVLLLCGAVFVSPSRVQPTESIVRFPNITN | pxEBNCP24-RBD |
| NO: 28 | LCPFGEVFNATRFASVYAWNRKRISNCVADYSVLYNSA | |
| | SFSTFKCYGVSPTKLNDLCFTNVYADSFVIRGDEVRQIA | |
| | PGQTGKIADYNYKLPDDFTGCVIAWNSNNLDSKVGGNY | |
| | NYLYRLFRKSNLKPFERDISTEIYQAGSTPCNGVEGFNC | |
| | YFPLQSYGFQPTNGVGYQPYRVVVLSFELLHAPATVCG | |
| | PKKSTNLVKNKCVNFGGGPPSVFIFPPKIKDVLMISLSPIV | |
| | TCVVVDVSEDDPDVQISWFVNNVEVHTAQTQTHREDYN | |
| | STLRVVSALPIQHQDWMSGKEFKCKVNNKDLPAPIERTI | |
| | SKPKGSVRAPQVYVLPPPEEEMTKKQVTLTCMVTDFMP | |
| | EDIYVEWTNNGKTELNYKNTEPVLDSDGSYFMYSKLRV | |
| | EKKNWVERNSYSCSVVHEGLHNHHTTKSFSRTPGK | |
| SEQ ID | MDAMKRGLCCVLLLCGAVFVSPSRVQPTESIVRFPNITN | pxEBNCP25-RBD |
| NO: 29 | LCPFGEVFNATRFASVYAWNRKRISNCVADYSVLYNSA | |
| | SFSTFKCYGVSPTKLNDLCFTNVYADSFVIRGDEVRQIA | |
| | PGQTGKIADYNYKLPDDFTGCVIAWNSNNLDSKVGGNY | |
| | NYLYRLFRKSNLKPFERDISTEIYQAGSTPCNGVEGFNC | |
| | YFPLQSYGFQPTNGVGYQPYRVVVLSFELLHAPATVCG | |
| | PKKSTNLVKNKCVNFGGGPKPLDGEYFTLQIRGRERFE | |
| | MFRELNEALELKDAQAGKEPGHHHHHHHH | |
| SEQ ID | MDAMKRGLCCVLLLCGAVFVSPSVEKGIYQTSNFRVQP | pxEBNCP29-RBD |
| NO: 30 | TESIVRFPNITNLCPFGEVFNATRFASVYAWNRKRISNCV | |
| | ADYSVLYNSASFSTFKCYGVSPTKLNDLCFTNVYADSFV | |
| | IRGDEVRQIAPGQTGKIADYNYKLPDDFTGCVIAWNSNN | |
| | LDSKVGGNYNYLYRLFRKSNLKPFERDISTEIYQAGSTP | |
| | CNGVEGFNCYFPLQSYGFQPTNGVGYQPYRVVVLSFEL | |
| | LHAPATVCGPKKSTNLVKNKCVNFNFNGLTGTGVLTES | |
| | NKGGGSHHHHHHHH | |
| SEQ ID | MDAMKRGLCCVLLLCGAVFVSPSITNLCPFGEVFNATRF | pxEBNCP30-RBD |
| NO: 31 | ASVYAWNRKRISNCVADYSVLYNSASFSTFKCYGVSPT | |
| | KLNDLCFTNVYADSFVIRGDEVRQIAPGQTGKIADYNYKL | |
| | PDDFTGCVIAWNSNNLDSKVGGNYNYLYRLFRKSNLKP | |
| | FERDISTEIYQAGSTPCNGVEGFNCYFPLQSYGFQPTNG | |
| | VGYQPYRVVVLSFELLGGGSHHHHHHHH | |
| SEQ ID | MDAMKRGLCCVLLLCGAVFVSPSITNLCPFGEVFNATRF | pxEBNCP31-RBD |
| NO: 32 | ASVYAWNRKRISNCVADYSVLYNSASFSTFKCYGVSPT | |
| | KLNDLCFTNVYADSFVIRGDEVRQIAPGQTGKIADYNYKL | |
| | PDDFTGCVIAWNSNNLDSKVGGNYNYLYRLFRKSNLKP | |
| | FERDISTEIYQAGSTPCNGVEGFNCYFPLQSYGFQPTNG | |
| | VGYQPYRVVVLSFELLHAPATVCGPKKSTNLVKNKCVNF | |
| | NFNGLTGTGVLTESNKGGGSHHHHHHHH | |
| SEQ ID | MDAMKRGLCCVLLLCGAVFVSPSASWFTALTQHGKEDL | pxENBEP32- |
| NO: 33 | KFPRGQGVPINTNSSPDDQIGYYRRATRRIRGGDGKMK | NucRBD |
| | DLSPRWYFYYLGTGPEAGLPYGANKDGIIWVATEGALNT | |
| | PKDHIGTRNPANNAAIVLQLPQGTTLPKGFYAGGSGGRV | |
| | QPTESIVRFPNITNLCPFGEVFNATRFASVYAWNRKRISN | |
| | CVADYSVLYNSASFSTFKCYGVSPTKLNDLCFTNVYADS | |
| | FVIRGDEVRQIAPGQTGKIADYNYKLPDDFTGCVIAWNS | |
| | NNLDSKVGGNYNYLYRLFRKSNLKPFERDISTEIYQAGS | |
| | TPCNGVEGFNCYFPLQSYGFQPTNGVGYQPYRVVVLSF | |
| | ELLHAPATVCGPKKSTNLVKNKCVNFGGGSHHHHHHHH | |
| | HH | |
| SEQ ID | MDAMKRGLCCVLLLCGAVFVSPSRVQPTESIVRFPNITN | pxENBEP33- |
| NO: 34 | LCPFGEVFNATRFASVYAWNRKRISNCVADYSVLYNSA | RBDNuc |
| | SFSTFKCYGVSPTKLNDLCFTNVYADSFVIRGDEVRQIA | |
| | PGQTGKIADYNYKLPDDFTGCVIAWNSNNLDSKVGGNY | |
| | NYLYRLFRKSNLKPFERDISTEIYQAGSTPCNGVEGFNC | |
| | YFPLQSYGFQPTNGVGYQPYRVVVLSFELLHAPATVCG | |
| | PKKSTNLVKNKCVNFGGSGGASWFTALTQHGKEDLKFP | |
| | RGQGVPINTNSSPDDQIGYYRRATRRIRGGDGKMKDLS | |
| | PRWYFYYLGTGPEAGLPYGANKDGIIWVATEGALNTPK | |
| | DHIGTRNPANNAAIVLQLPQGTTLPKGFYAGGGSHHHH | |
| | HHHHHH | |
| SEQ ID | MDAMKRGLCCVLLLCGAVFVSPSASWFTALTQHGKEDL | pxENBEP34- |
| NO: 35 | KFPRGQGVPINTNSSPDDQIGYYRRATRRIRGGDGKMK | NucRBD |
| | DLSPRWYFYYLGTGPEAGLPYGANKDGIIWVATEGALNT | |
| | PKDHIGTRNPANNAAIVLQLPQGTTLPKGFYAGGSGGG | |
| | GSAEASKKNVTQAFGRRGPEQTQGNFGDQELIRQGTD | |
| | YKHWPQIAQFAPSASAFFGMSRIGMEVTPSGTWLTYTG | |
| | AIKLDDKDPNFKDQVILLNKHIDAYKTFGGSGGRVQPTE | |
| | SIVRFPNITNLCPFGEVFNATRFASVYAWNRKRISNCVA | |
| | DYSVLYNSASFSTFKCYGVSPTKLNDLCFTNVYADSFVI | |
| | RGDEVRQIAPGQTGKIADYNYKLPDDFTGCVIAWNSNNL | |
| | DSKVGGNYNYLYRLFRKSNLKPFERDISTEIYQAGSTPC | |
| | NGVEGFNCYFPLQSYGFQPTNGVGYQPYRVVVLSFELL | |
| | HAPATVCGPKKSTNLVKNKCVNFGGGSHHHHHHHHHH | |
| SEQ ID | MDAMKRGLCCVLLLCGAVFVSPSRVQPTESIVRFPNITN | pxENBEP35- |
| NO: 36 | LCPFGEVFNATRFASVYAWNRKRISNCVADYSVLYNSA | RBDNuc |
| | SFSTFKCYGVSPTKLNDLCFTNVYADSFVIRGDEVRQIA | |
| | PGQTGKIADYNYKLPDDFTGCVIAWNSNNLDSKVGGNY | |
| | NYLYRLFRKSNLKPFERDISTEIYQAGSTPCNGVEGFNC | |
| | YFPLQSYGFQPTNGVGYQPYRVVVLSFELLHAPATVCG | |
| | PKKSTNLVKNKCVNFGGSGGASWFTALTQHGKEDLKFP | |
| | RGQGVPINTNSSPDDQIGYYRRATRRIRGGDGKMKDLS | |
| | PRWYFYYLGTGPEAGLPYGANKDGIIWVATEGALNTPK | |
| | DHIGTRNPANNAAIVLQLPQGTTLPKGFYAGGSGGGGS | |
| | AEASKKNVTQAFGRRGPEQTQGNFGDQELIRQGTDYK | |
| | HWPQIAQFAPSASAFFGMSRIGMEVTPSGTWLTYTGAIK | |
| | LDDKDPNFKDQVILLNKHIDAYKTFGGGSHHHHHHHHH | |
| | H | |
| SEQ ID | MDAMKRGLCCVLLLCGAVFVSPSVEKGIYQTSNFRVQP | pxEBNCP26-RBD |
| NO: 37 | TESIVRFPNITNLCPFGEVFNATRFASVYAWNRKRISNCV | |
| | ADYSVLYNSASFSTFKCYGVSPTKLNDLCFTNVYADSFV | |
| | IRGDEVRQIAPGQTGKIADYNYKLPDDFTGCVIAWNSNN | |
| | LDSKVGGNYNYLYRLFRKSNLKPFERDISTEIYQAGSTP | |
| | CNGVEGFNCYFPLQSYGFQPTNGVGYQPYRVVVLSFEL | |
| | LHAPATVCGPKKSTNLVKNKCVNFNFNGLTGTGVLTES | |
| | NKGGGSQLTPTFYDNSCPNVSNIVRDTIVNELRSDPRIA | |
| | ASILRLHFHDCFVNGCDASILLDNTTSFRTEKDAFGNANS | |
| | ARGFPVIDRMKAAVESACPRTVSCADLLTIAAQQSVTLA | |
| | GGPSWRVPLGRRDSLQAFLDLANANLPAPFFTLPQLKD | |
| | SFRNVGLNRSSDLVALSGGHTFGKNQCRFIMDRLYNFS | |
| | NTGLPDPTLNTTYLQTLRGLCPLNGNLSALVDFDLRTPTI | |
| | FDNKYYVNLEEQKGLIQSDQELFSSPNATDTIPLVRSFAN | |
| | STQTFFNAFVEAMDRMGNITPLTGTQGQIRLNCRVVNS | |
| | NSGGGSHHHHHHHH | |
| SEQ ID | MDAMKRGLCCVLLLCGAVFVSPSHHHHHHHHGGGSQL | pxEBNCP27-RBD |
| NO: 38 | TPTFYDNSCPNVSNIVRDTIVNELRSDPRIAASILRLHFHD | |
| | CFVNGCDASILLDNTTSFRTEKDAFGNANSARGFPVIDR | |
| | MKAAVESACPRTVSCADLLTIAAQQSVTLAGGPSWRVP | |
| | LGRRDSLQAFLDLANANLPAPFFTLPQLKDSFRNVGLNR | |
| | SSDLVALSGGHTFGKNQCRFIMDRLYNFSNTGLPDPTLN | |
| | TTYLQTLRGLCPLNGNLSALVDFDLRTPTIFDNKYYVNLE | |
| | EQKGLIQSDQELFSSPNATDTIPLVRSFANSTQTFFNAFV | |
| | EAMDRMGNITPLTGTQGQIRLNCRVVNSNSGGGSVEKG | |
| | IYQTSNFRVQPTESIVRFPNITNLCPFGEVFNATRFASVY | |
| | AWNRKRISNCVADYSVLYNSASFSTFKCYGVSPTKLNDL | |
| | CFTNVYADSFVIRGDEVRQIAPGQTGKIADYNYKLPDDF | |
| | TGCVIAWNSNNLDSKVGGNYNYLYRLFRKSNLKPFERDI | |
| | STEIYQAGSTPCNGVEGFNCYFPLQSYGFQPTNGVGYQ | |
| | PYRVVVLSFELLHAPATVCGPKKSTNLVKNKCVNFNFNG | |
| | LTGTGVLTESNK | |
| SEQ ID | MFVFLVLLPLVSSQCHHHHHHHHGGGSENLYFQRVQPT | pxENB36- |
| NO: 39 | ESIVRFPNITNLCPFGEVFNATRFASVYAWNRKRISNCVA | H8RBDgpp53 |
| | DYSVLYNSASFSTFKCYGVSPTKLNDLCFTNVYADSFVI | |
| | RGDEVRQIAPGQTGKIADYNYKLPDDFTGCVIAWNSNNL | |
| | DSKVGGNYNYLYRLFRKSNLKPFERDISTEIYQAGSTPC | |
| | NGVEGFNCYFPLQSYGFQPTNGVGYQPYRVVVLSFELL | |
| | HAPATVCGPKKSTNLVKNKCVNFGGGPKPLDGEYFTLQI | |
| | RGRERFEMFRELNEALELKDAQAGKEPG | |
| SEQ ID | MFVFLVLLPLVSSQCHHHHHHHHGGGSENLYFQRVQPT | pxENB37- |
| NO: 40 | ESIVRFPNITNLCPFGEVFNATRFASVYAWNRKRISNCVA | H8RBDgsp53 |
| | DYSVLYNSASFSTFKCYGVSPTKLNDLCFTNVYADSFVI | |
| | RGDEVRQIAPGQTGKIADYNYKLPDDFTGCVIAWNSNNL | |
| | DSKVGGNYNYLYRLFRKSNLKPFERDISTEIYQAGSTPC | |
| | NGVEGFNCYFPLQSYGFQPTNGVGYQPYRVVVLSFELL | |
| | HAPATVCGPKKSTNLVKNKCVNFGGGSKPLDGEYFTLQI | |
| | RGRERFEMFRELNEALELKDAQAGKEPG | |
| SEQ ID | MFVFLVLLPLVSSQCRVQPTESIVRFPNITNLCPFGEVFN | pxENB38- |
| NO: 41 | ATRFASVYAWNRKRISNCVADYSVLYNSASFSTFKCYG | RBDgpp53H8 |
| | VSPTKLNDLCFTNVYADSFVIRGDEVRQIAPGQTGKIADY | |
| | NYKLPDDFTGCVIAWNSNNLDSKVGGNYNYLYRLFRKS | |
| | NLKPFERDISTEIYQAGSTPCNGVEGFNCYFPLQSYGFQ | |
| | PTNGVGYQPYRVVVLSFELLHAPATVCGPKKSTNLVKN | |
| | KCVNFGGGPKPLDGEYFTLQIRGRERFEMFRELNEALEL | |
| | KDAQAGKEPGHHHHHHHH | |
| SEQ ID | MFVFLVLLPLVSSQCRVQPTESIVRFPNITNLCPFGEVFN | pxENB39- |
| NO: 42 | ATRFASVYAWNRKRISNCVADYSVLYNSASFSTFKCYG | RBDgsp53H8 |
| | VSPTKLNDLCFTNVYADSFVIRGDEVRQIAPGQTGKIADY | |
| | NYKLPDDFTGCVIAWNSNNLDSKVGGNYNYLYRLFRKS | |
| | NLKPFERDISTEIYQAGSTPCNGVEGFNCYFPLQSYGFQ | |
| | PTNGVGYQPYRVVVLSFELLHAPATVCGPKKSTNLVKN | |
| | KCVNFGGGSKPLDGEYFTLQIRGRERFEMFRELNEALEL | |
| | KDAQAGKEPGHHHHHHHH | |
| SEQ ID | MFVFLVLLPLVSSQCHHHHHHHHGGGSENLYFQRVQPT | pxENB40-H8RBDFc |
| NO: 43 | ESIVRFPNITNLCPFGEVFNATRFASVYAWNRKRISNCVA | |
| | DYSVLYNSASFSTFKCYGVSPTKLNDLCFTNVYADSFVI | |
| | RGDEVRQIAPGQTGKIADYNYKLPDDFTGCVIAWNSNNL | |
| | DSKVGGNYNYLYRLFRKSNLKPFERDISTEIYQAGSTPC | |
| | NGVEGFNCYFPLQSYGFQPTNGVGYQPYRVVVLSFELL | |
| | HAPATVCGPKKSTNLVKNKCVNFVPRDCGCKPCICTVP | |
| | EVSSVFIFPPKPKDVLTITLTPKVTCVVVDISKDDPEVQFS | |
| | WFVDDVEVHTAQTQPREEQFNSTFRSVSELPIMHQDWL | |
| | NGKEFKCRVNSAAFPAPIEKTISKTKGRPKAPQVYTIPPP | |
| | KEQMAKDKVSLTCMITDFFPEDITVEWQWNGQPAENYK | |
| | NTQPIMNTNGSYFVYSKLNVQKSNWEAGNTFTCSVLHE | |
| | GLHNHHTEKSLSHSPGK | |
| SEQ ID | MFVFLVLLPLVSSQCHHHHHHHHGGGSENLYFQRVQPT | pxENB41- |
| NO: 44 | ESIVRFPNITNLCPFGEVFNATRFASVYAWNRKRISNCVA | H8RBDFcSBP |
| | DYSVLYNSASFSTFKCYGVSPTKLNDLCFTNVYADSFVI | |
| | RGDEVRQIAPGQTGKIADYNYKLPDDFTGCVIAWNSNNL | |
| | DSKVGGNYNYLYRLFRKSNLKPFERDISTEIYQAGSTPC | |
| | NGVEGFNCYFPLQSYGFQPTNGVGYQPYRVVVLSFELL | |
| | HAPATVCGPKKSTNLVKNKCVNFVPRDCGCKPCICTVP | |
| | EVSSVFIFPPKPKDVLTITLTPKVTCVVVDISKDDPEVQFS | |
| | WFVDDVEVHTAQTQPREEQFNSTFRSVSELPIMHQDWL | |
| | NGKEFKCRVNSAAFPAPIEKTISKTKGRPKAPQVYTIPPP | |
| | KEQMAKDKVSLTCMITDFFPEDITVEWQWNGQPAENYK | |
| | NTQPIMNTNGSYFVYSKLNVQKSNWEAGNTFTCSVLHE | |
| | GLHNHHTEKSLSHSPGKGGGSMDEKTTGWRGGHVVE | |
| | GLAGELEQLRARLEHHPQGQREP | |
| SEQ ID | MFVFLVLLPLVSSQCHHHHHHHHGGGSENLYFQRVQPT | pxENB42- |
| NO: 45 | ESIVRFPNITNLCPFGEVFNATRFASVYAWNRKRISNCVA | H8RBDFcHG |
| | DYSVLYNSASFSTFKCYGVSPTKLNDLCFTNVYADSFVI | |
| | RGDEVRQIAPGQTGKIADYNYKLPDDFTGCVIAWNSNNL | |
| | DSKVGGNYNYLYRLFRKSNLKPFERDISTEIYQAGSTPC | |
| | NGVEGFNCYFPLQSYGFQPTNGVGYQPYRVVVLSFELL | |
| | HAPATVCGPKKSTNLVKNKCVNFVPRDCGCKPCICT | |
| SEQ ID | MFVFLVLLPLVSSQCHHHHHHHHGGGSENLYFQRVQPT | pxENB43- |
| NO: 46 | ESIVRFPNITNLCPFGEVFNATRFASVYAWNRKRISNCVA | RBDFcHGSBP |
| | DYSVLYNSASFSTFKCYGVSPTKLNDLCFTNVYADSFVI | |
| | RGDEVRQIAPGQTGKIADYNYKLPDDFTGCVIAWNSNNL | |
| | DSKVGGNYNYLYRLFRKSNLKPFERDISTEIYQAGSTPC | |
| | NGVEGFNCYFPLQSYGFQPTNGVGYQPYRVVVLSFELL | |
| | HAPATVCGPKKSTNLVKNKCVNFVPRDCGCKPCICTGG | |
| | GSMDEKTTGWRGGHVVEGLAGELEQLRARLEHHPQGQ | |
| | REP | |
| SEQ ID | MFVFLVLLPLVSSQCHHHHHHHHGGGSENLYFQRVQPT | pxENB44- |
| NO: 47 | ESIVRFPNITNLCPFGEVFNATRFASVYAWNRKRISNCVA | H8RBDRBD |
| | DYSVLYNSASFSTFKCYGVSPTKLNDLCFTNVYADSFVI | |
| | RGDEVRQIAPGQTGKIADYNYKLPDDFTGCVIAWNSNNL | |
| | DSKVGGNYNYLYRLFRKSNLKPFERDISTEIYQAGSTPC | |
| | NGVEGFNCYFPLQSYGFQPTNGVGYQPYRVVVLSFELL | |
| | HAPATVCGPKKSTNLVKNKCVNFRVQPTESIVRFPNITN | |
| | LCPFGEVFNATRFASVYAWNRKRISNCVADYSVLYNSA | |
| | SFSTFKCYGVSPTKLNDLCFTNVYADSFVIRGDEVRQIA | |
| | PGQTGKIADYNYKLPDDFTGCVIAWNSNNLDSKVGGNY | |
| | NYLYRLFRKSNLKPFERDISTEIYQAGSTPCNGVEGFNC | |
| | YFPLQSYGFQPTNGVGYQPYRVVVLSFELLHAPATVCG | |
| | PKKSTNLVKNKCVNF | |
| SEQ ID | MFVFLVLLPLVSSQCRVQPTESIVRFPNITNLCPFGEVFN | pxENB46- |
| NO: 48 | ATRFASVYAWNRKRISNCVADYSVLYNSASFSTFKCYG | RBDRBDH8 |
| | VSPTKLNDLCFTNVYADSFVIRGDEVRQIAPGQTGKIADY | |
| | NYKLPDDFTGCVIAWNSNNLDSKVGGNYNYLYRLFRKS | |
| | NLKPFERDISTEIYQAGSTPCNGVEGFNCYFPLQSYGFQ | |
| | PTNGVGYQPYRVVVLSFELLHAPATVCGPKKSTNLVKN | |
| | KCVNFRVQPTESIVRFPNITNLCPFGEVFNATRFASVYA | |
| | WNRKRISNCVADYSVLYNSASFSTFKCYGVSPTKLNDLC | |
| | FTNVYADSFVIRGDEVRQIAPGQTGKIADYNYKLPDDFT | |
| | GCVIAWNSNNLDSKVGGNYNYLYRLFRKSNLKPFERDIS | |
| | TEIYQAGSTPCNGVEGFNCYFPLQSYGFQPTNGVGYQP | |
| | YRVVVLSFELLHAPATVCGPKKSTNLVKNKCVNFGGGS | |
| | HHHHHHHH | |
| SEQ ID | MFVFLVLLPLVSSQCHHHHHHHHGGGSENLYFQRVQPT | pxENB48- |
| NO: 49 | ESIVRFPNITNLCPFGEVFNATRFASVYAWNRKRISNCVA | H8RBDSBP |
| | DYSVLYNSASFSTFKCYGVSPTKLNDLCFTNVYADSFVI | |
| | RGDEVRQIAPGQTGKIADYNYKLPDDFTGCVIAWNSNNL | |
| | DSKVGGNYNYLYRLFRKSNLKPFERDISTEIYQAGSTPC | |
| | NGVEGFNCYFPLQSYGFQPTNGVGYQPYRVVVLSFELL | |
| | HAPATVCGPKKSTNLVKNKCVNFGGGSMDEKTTGWRG | |
| | GHVVEGLAGELEQLRARLEHHPQGQREP | |
| SEQ ID | MFVFLVLLPLVSSQHHHHHHHHGGGSENLYFQRVQPTE | pxENB14-RBD- |
| NO: 50 | SIVRFPNITNLCPFGEVFNATRFASVYAWNRKRISNCVA | B.1.1.7 |
| | DYSVLYNSASFSTFKCYGVSPTKLNDLCFTNVYADSFVI | |
| | RGDEVRQIAPGQTGKIADYNYKLPDDFTGCVIAWNSNNL | |
| | DSKVGGNYNYLYRLFRKSNLKPFERDISTEIYQAGSTPC | |
| | NGVEGFNCYFPLQSYGFQPTYGVGYQPYRVVVLSFELL | |
| | HAPATVCGPKKSTNLVKNKCVNF | |
| SEQ ID | MFVFLVLLPLVSSQHHHHHHHHGGGSENLYFQRVQPTE | pxENB14-RBD- |
| NO: 51 | SIVRFPNITNLCPFGEVFNATRFASVYAWNRKRISNCVA | B.1.351 |
| | DYSVLYNSASFSTFKCYGVSPTKLNDLCFTNVYADSFVI | |
| | RGDEVRQIAPGQTGNIADYNYKLPDDFTGCVIAWNSNNL | |
| | DSKVGGNYNYLYRLFRKSNLKPFERDISTEIYQAGSTPC | |
| | NGVKGFNCYFPLQSYGFQPTYGVGYQPYRVVVLSFELL | |
| | HAPATVCGPKKSTNLVKNKCVNF | |
| SEQ ID | MFVFLVLLPLVSSQHHHHHHHHGGGSENLYFQRVQPTE | pxENB14-RBD- |
| NO: 52 | SIVRFPNITNLCPFGEVFNATRFASVYAWNRKRISNCVA | B.1.617.2 |
| | DYSVLYNSASFSTFKCYGVSPTKLNDLCFTNVYADSFVI | |
| | RGDEVRQIAPGQTGKIADYNYKLPDDFTGCVIAWNSNNL | |
| | DSKVGGNYNYRYRLFRKSNLKPFERDISTEIYQAGSKPC | |
| | NGVEGFNCYFPLQSYGFQPTNGVGYQPYRVVVLSFELL | |
| | HAPATVCGPKKSTNLVKNKCVNF | |
| SEQ ID | MFVFLVLLPLVSSQHHHHHHHHGGGSENLYFQRVQPTE | pxENB14-RBD- |
| NO: 53 | SIVRFPNITNLCPFGEVFNATRFASVYAWNRKRISNCVA | B.1.427 |
| | DYSVLYNSASFSTFKCYGVSPTKLNDLCFTNVYADSFVI | |
| | RGDEVRQIAPGQTGKIADYNYKLPDDFTGCVIAWNSNNL | |
| | DSKVGGNYNYRYRLFRKSNLKPFERDISTEIYQAGSTPC | |
| | NGVEGFNCYFPLQSYGFQPTNGVGYQPYRVVVLSFELL | |
| | HAPATVCGPKKSTNLVKNKCVNF | |
| SEQ ID | MFVFLVLLPLVSSQHHHHHHHHGGGSENLYFQRVQPTE | pxENB14-RBD-P.1 |
| NO: 54 | SIVRFPNITNLCPFGEVFNATRFASVYAWNRKRISNCVA | |
| | DYSVLYNSASFSTFKCYGVSPTKLNDLCFTNVYADSFVI | |
| | RGDEVRQIAPGQTGTIADYNYKLPDDFTGCVIAWNSNNL | |
| | DSKVGGNYNYLYRLFRKSNLKPFERDISTEIYQAGSTPC | |
| | NGVKGFNCYFPLQSYGFQPTYGVGYQPYRVVVLSFELL | |
| | HAPATVCGPKKSTNLVKNKCVNF | |
| SEQ ID | MFVFLVLLPLVSSQCRVQPTESIVRFPNITNLCPFGEVEN | pxENB46-RBD2- |
| NO: 55 | ATRFASVYAWNRKRISNCVADYSVLYNSASFSTFKCYG | B.1.1.7 |
| | VSPTKLNDLCFTNVYADSFVIRGDEVRQIAPGQTGKIADY | |
| | NYKLPDDFTGCVIAWNSNNLDSKVGGNYNYLYRLFRKS | |
| | NLKPFERDISTEIYQAGSTPCNGVEGFNCYFPLQSYGFQ | |
| | PTYGVGYQPYRVVVLSFELLHAPATVCGPKKSTNLVKNK | |
| | CVNFRVQPTESIVRFPNITNLCPFGEVFNATRFASVYAW | |
| | NRKRISNCVADYSVLYNSASFSTFKCYGVSPTKLNDLCF | |
| | TNVYADSFVIRGDEVRQIAPGQTGKIADYNYKLPDDFTG | |
| | CVIAWNSNNLDSKVGGNYNYLYRLFRKSNLKPFERDIST | |
| | EIYQAGSTPCNGVEGFNCYFPLQSYGFQPTYGVGYQPY | |
| | RVVVLSFELLHAPATVCGPKKSTNLVKNKCVNFGGGSH | |
| | HHHHHHH | |
| SEQ ID | MFVFLVLLPLVSSQCRVQPTESIVRFPNITNLCPFGEVEN | pxENB46-RBD2- |
| NO: 56 | ATRFASVYAWNRKRISNCVADYSVLYNSASFSTFKCYG | B.1.351 |
| | VSPTKLNDLCFTNVYADSFVIRGDEVRQIAPGQTGNIAD | |
| | YNYKLPDDFTGCVIAWNSNNLDSKVGGNYNYLYRLFRK | |
| | SNLKPFERDISTEIYQAGSTPCNGVKGFNCYFPLQSYGF | |
| | QPTYGVGYQPYRVVVLSFELLHAPATVCGPKKSTNLVK | |
| | NKCVNFRVQPTESIVRFPNITNLCPFGEVFNATRFASVY | |
| | AWNRKRISNCVADYSVLYNSASFSTFKCYGVSPTKLNDL | |
| | CFTNVYADSFVIRGDEVRQIAPGQTGNIADYNYKLPDDF | |
| | TGCVIAWNSNNLDSKVGGNYNYLYRLFRKSNLKPFERDI | |
| | STEIYQAGSTPCNGVKGFNCYFPLQSYGFQPTYGVGYQ | |
| | PYRVVVLSFELLHAPATVCGPKKSTNLVKNKCVNFGGG | |
| | SHHHHHHHH | |
| SEQ ID | MFVFLVLLPLVSSQCRVQPTESIVRFPNITNLCPFGEVFN | pxENB46-RBD2- |
| NO: 57 | ATRFASVYAWNRKRISNCVADYSVLYNSASFSTFKCYG | B.1.617.2 |
| | VSPTKLNDLCFTNVYADSFVIRGDEVRQIAPGQTGKIADY | |
| | NYKLPDDFTGCVIAWNSNNLDSKVGGNYNYRYRLFRKS | |
| | NLKPFERDISTEIYQAGSKPCNGVEGFNCYFPLQSYGFQ | |
| | PTNGVGYQPYRVVVLSFELLHAPATVCGPKKSTNLVKN | |
| | KCVNFRVQPTESIVRFPNITNLCPFGEVFNATRFASVYA | |
| | WNRKRISNCVADYSVLYNSASFSTFKCYGVSPTKLNDLC | |
| | FTNVYADSFVIRGDEVRQIAPGQTGKIADYNYKLPDDFT | |
| | GCVIAWNSNNLDSKVGGNYNYRYRLFRKSNLKPFERDI | |
| | STEIYQAGSKPCNGVEGFNCYFPLQSYGFQPTNGVGYQ | |
| | PYRVVVLSFELLHAPATVCGPKKSTNLVKNKCVNFGGG | |
| | SHHHHHHHH | |

1. A fusion protein comprising a SARS-COV-2 receptor binding domain (RBD) of the SARS-COV-2 spike protein or a fragment thereof, and a N-terminal signal peptide, and at least one of a polyhistidine tag, linker, an oligomerization tag, a region in spike protein outside RBD, a streptavidin binding peptide, a horseradish peroxidase binding domain or a protease cleavage site.

2. The fusion protein, according to claim 1, wherein the N-terminal signal peptide is selected from a spike endogenous signal peptide, a tissue plasminogen activator (tPa).

3. The fusion protein, according to claim 1 or 2, wherein the N-terminal signal peptide has an amino acid sequence selected from SEQ ID NO: 1 and SEQ ID NO:2.

4. The fusion protein, according to any of the preceding claims, wherein the polyhistidine tag consists of 8 or 10 histidine residues.

5. The fusion protein, according to claim 4, wherein the polyhistidine tag has an amino acid sequence selected from SEQ ID NO:7 and SEQ ID NO:8.

6. The fusion protein, according to claim 1, wherein the oligomerization tag is selected from a murine IgG1-Fc (CH2, CH3 only), a murine IgG1-Fc dimerization domain, a murine IgG-2a-Fc (CH2, CH3 only), a murine IgG-2a-Fc dimerization domain, a p53 tetramerization domain, a SARS-COV-2 nucleocapsid N-terminal domain and a SARS-COV-2 nucleocapsid C-terminal domain.

7. The fusion protein, according to claim 6, wherein the oligomerization tag has an amino acid sequence selected SEQ ID NO:9, SEQ ID NO:10, SEQ ID NO:11, SEQ ID NO:12, SEQ ID NO:13, SEQ ID NO: 14 and SEQ ID NO:15.

8. The fusion protein, according to claim 1, wherein the linker is a flexible linker.

9. The fusion protein, according to claim 8, wherein the linker has an amino acid sequence selected from SEQ ID NO:3, SEQ ID NO:4, SEQ ID NO:5 and SEQ ID NO:6.

10. The fusion protein, according to claim 1, wherein the Streptavidin binding peptide tag is or comprises SEQ ID NO:17.

11. The fusion protein, according to claim 1, wherein the horseradish peroxidase binding domain has an amino acid sequence selected from SEQ ID NO:18.

12. The fusion protein, according to claim 1, wherein the protease cleavage site is a tobacco etch virus cleavage site (TEV).

13. The fusion protein, according to claim 12, wherein the protease cleavage site has an amino acid sequence selected from SEQ ID NO:19.

14. The fusion protein, according to claim 1, wherein the receptor binding domain (RBD) of the SARS-COV-2 spike protein or a fragment thereof has an amino acid sequence of at least 90% sequence identity with SEQ ID NO:20.

15. The fusion protein, according to claim 1, wherein the fusion protein has an amino acid sequence of at least 90% sequence identity with SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56 or SEQ ID NO:57.

16. The fusion protein, according to claim 1, wherein the SARS-COV-2 RBD protein comprises a mutation in one or more of the following positions: G404, A475, T478, N481, G485, F490, Q493, G496, Q498, N501, or V503.

17. A cell, comprising the fusion protein according to claim 1.

18. A nucleic acid comprising a nucleotide sequence encoding the fusion protein according to claim 1, a promoter operably linked to the nucleotide sequence and a selectable marker.

19. A cell comprising the nucleic acid of claim 18.

20. A composition comprising the fusion protein of claim 1, and a solid support, wherein the fusion protein is covalently or non-covalently bound to the solid support.
